(19) United States
(12) Patent Application Publication
(10) Pub. No.: US 2017/0156695 A1
(43) Pub. Date: Jun. 8, 2017

(54) ULTRASONIC PROBE UNIT, ULTRASONIC PROBE, AND ULTRASONIC APPARATUS

(71) Applicant: Seiko Epson Corporation, Tokyo (JP)

(72) Inventors: Tomoaki NAKAMURA, Chino (JP); Kanechika KIYOSE, Matsumoto (JP)

(21) Appl. No.: 15/364,719

(22) Filed: Nov. 30, 2016

(30) Foreign Application Priority Data
Dec. 2, 2015 (JP) 2015-235448

(51) Int. Cl. A61B 8/00; A61B 8/14

(52) U.S. Cl. CPC A61B 8/4461; A61B 8/4236; A61B 8/14; A61B 8/4427; A61B 8/4494

(57) ABSTRACT

An ultrasonic probe unit is provided with an ultrasonic probe, and a fixation section adapted to fix the ultrasonic probe to a living body, the ultrasonic probe includes an ultrasonic device adapted to perform transmission and reception of an ultrasonic wave, a probe-side magnet, and a displacement mechanism adapted to adjust a relative positional relationship between the ultrasonic device and the probe-side magnet, and the fixation section includes a fixation surface to be fixed to the living body, and a fixation section-side magnet a relative position of which to the probe-side magnet is fixed to thereby fix the ultrasonic probe to the living body.

### BACKGROUND

1. Technical Field

The present invention relates to an ultrasonic probe unit, an ultrasonic probe, and an ultrasonic apparatus.

2. Related Art

In the past, there has been known an ultrasonic probe unit for transmitting an ultrasonic wave to the inside of a measurement object (e.g., a living body) with an ultrasonic probe fixedly adhering to the measurement object (see, e.g., JP-A-2011-62373 (Document 1)).

In Document 1, there is a description related to an ultrasonic oscillation unit equipped with an ultrasonic oscillator and a fixation device. The fixation device of the ultrasonic oscillation unit has an oscillator support device for supporting an ultrasonic oscillator, and a body surface-side fixation device to be fixed to a body surface. The oscillator support device has a cylinder part for supporting the ultrasonic oscillator, and a flange part disposed on one end (body surface side) of the cylinder part, and by connecting the flange part to the body surface-side fixation device fixed to the body surface, the ultrasonic oscillator is fixed to the living body.

Further, as a detaching/attaching configuration between the body surface-side fixation device and the flange part, there are cited (i) a configuration in which the body surface-side fixation device is provided with an iron paper, the flange part is formed of a magnet, and the flange part and the body surface-side fixation device are connected with the magnetic force, (ii) a configuration in which a body surface side and a flange part side of the body surface-side fixation device are each formed of an adhesive surface, and the flange part formed of resin is made to fixedly adhere to the adhesive surface of the body surface-side fixation device, and (iii) a configuration in which a female engagement device provided to the flange part is engaged with a male engagement device provided to the body surface-side fixation device to thereby achieve the fixation.

Incidentally, in the device described in Document 1 described above, when the ultrasonic probe (the ultrasonic oscillator) is fixed to the body surface with the fixation device, the position where the ultrasonic wave is transmitted is fixed. However, in some cases, the region to be the measurement object is shifted from the direction (e.g., immediately below the ultrasonic probe) in which the ultrasonic wave is transmitted from the ultrasonic probe, and in this case, in the device described in Document 1 described above, it is necessary to fix the ultrasonic probe again to a correct position. Specifically, it is necessary to detach the oscillator support device from the body surface-side fixation device, then detach the body surface-side fixation device from the body surface, then stick the body surface-side fixation device to a correct position, and then attach the oscillator support device. Further, on this occasion, if the direction in which the ultrasonic wave is transmitted from the ultrasonic probe departs from the region of the measurement object, it is necessary to repeat the sequence described above to adjust the position. Therefore, in the fixation configuration of the ultrasonic probe in the related art, there is a problem that the fixation configuration is accompanied by the extremely cumbersome operation.

### SUMMARY

An advantage of some aspects of the invention is to provide an ultrasonic probe unit, an ultrasonic probe, and an ultrasonic apparatus allowing a user to easily fix the ultrasonic probe to the object at a desired position.

An ultrasonic probe unit according to an application example of the invention includes an ultrasonic probe, and a fixation section adapted to fix the ultrasonic probe to an object, the ultrasonic probe includes an ultrasonic device adapted to perform at least one of transmission and reception of an ultrasonic wave, a first fixation section, and an adjustment section adapted to adjust a relative positional relationship between the ultrasonic device and the first fixation section, and the fixation section includes a fixation surface to be fixed to the object, and a second fixation section a relative position of which to the first fixation section is fixed to thereby fix the ultrasonic probe to the object.

In this application example, the ultrasonic probe unit is provided with the fixation section and the ultrasonic probe. By fixing the fixation surface to the object, the fixation section is fixed to the object. In the ultrasonic probe, by fixing the relative position between the first fixation section provided to the ultrasonic probe and the second fixation section provided to the fixation section, the ultrasonic probe is fixed to the object. Here, the fixing method of the relative position between the first fixation section and the second fixation section is not particularly limited, and there can be cited a configuration of, for example, using a magnet as either one of the first fixation section and the second fixation section, and using a magnet or a magnetic material as the other. Besides the above, it is also possible to adopt a configuration of using a male engagement section as the first fixation section, using a female engagement section as the second fixation section, and making these sections engage with each other, or it is also possible to adopt a configuration of fixing by bonding (adhesively) the first fixation section to the second fixation section.

Further, in this application example, the ultrasonic probe is provided with the adjustment section for adjusting the relative positional relationship between the ultrasonic device and the first fixation section. As described above, when fixing the ultrasonic probe to the object, the relative position between the first fixation section and the second fixation section is fixed. Therefore, if the relative positional relationship between the ultrasonic device and the first fixation section is adjusted by the adjustment section, the relative position of the ultrasonic device to the object changes.

Therefore, even in the case in which the transmission direction (or the reception direction of the ultrasonic wave) of the ultrasonic wave is shifted from the transmission target (or the transmission (reflection) position of the ultrasonic wave to be received) of the ultrasonic wave in the object when fixing the fixation section to the object, and fixing the ultrasonic probe to the fixation section, it is possible to easily adjust the position of the ultrasonic device relatively to the object using the adjustment section. Therefore, in this application example, it is possible to easily fix the ultrasonic probe to the desired position for the object.

In the ultrasonic probe unit according to the application example, it is preferable that the adjustment section has a displacement mechanism adapted to displace the first fixation section relatively to the ultrasonic device.

In the application example with this configuration, the first fixation section is displaced relatively to the ultrasonic device using the displacement mechanism. Thus, similarly to the application example described above, even in the case in which the transmission direction (or the reception direction of the ultrasonic wave) of the ultrasonic wave is shifted from the transmission target (or the transmission (reflection) position of the ultrasonic wave to be received) of the ultrasonic wave in the object, it is possible to easily adjust the relative position of the ultrasonic device to the object to an appropriate position by relatively displacing the first fixation section to the ultrasonic device using the displacement mechanism.

In the ultrasonic probe unit according to the application example, it is preferable that the ultrasonic device has a device surface, which is at least one of a surface adapted to transmit the ultrasonic wave and a surface adapted to receive the ultrasonic wave, and the displacement mechanism displaces the first fixation section relatively to the ultrasonic device in a plane parallel to the device surface.

In the application example with this configuration, the displacement mechanism relatively displaces the ultrasonic device and the first fixation section to each other in a plane parallel to the device surface. Therefore, even in the case in which the positional relationship between the ultrasonic device and the first fixation section is adjusted by the displacement mechanism, the distance between the ultrasonic device and the object is not changed. Therefore, the distance (the depth) between the ultrasonic device and the transmission target (or the transmission (reflection) position of the ultrasonic wave to be received) of the ultrasonic wave in the object is not changed, and the transmission process and the reception process of the ultrasonic wave high in accuracy can be performed.

In the ultrasonic probe unit according to the application example, it is preferable that the ultrasonic device has a one-dimensional array structure in which a plurality of ultrasonic transducers is arranged along a first direction parallel to the device surface, and the displacement mechanism displaces the first fixation section relatively to the ultrasonic device along a second direction crossing the first direction.

In the application example with this configuration, the ultrasonic device has the ultrasonic transducers arranged in the first direction. In such a configuration, when transmitting the ultrasonic wave, for example, by controlling the transmission timings of the ultrasonic waves of the respective ultrasonic transducers arranged along the first direction, it becomes possible to control the transmission direction of the ultrasonic wave in a plane (a scan plane) including the first direction, in which the ultrasonic transducers are arranged, and perpendicular to the device surface. In contrast, in the second direction crossing the first direction, even by controlling the transmission timings of the ultrasonic waves of the respective ultrasonic transducers, the transmission direction of the ultrasonic wave cannot be changed. Therefore, in the ultrasonic device having the one-dimensional array structure, in the case in which the transmission direction of the ultrasonic wave, and the transmission target of the ultrasonic wave in the object are shifted from each other along the second direction, it becomes difficult to correct the shift only by the transmission control of each of the ultrasonic transducers. In contrast, in the application example, the first fixation section and the ultrasonic device are displaced along the second direction relatively to each other using the displacement mechanism, and thus, the scan plane can be displaced along the second direction. Therefore, as described above, even in the case in which the transmission direction of the ultrasonic wave and the transmission target of the ultrasonic wave in the object are shifted from each other along the second direction, the shift can easily be corrected by the displacement mechanism.

In the ultrasonic probe unit according to the application example, it is preferable that the ultrasonic device has a device surface, which is at least one of a surface adapted to transmit the ultrasonic wave and a surface adapted to receive the ultrasonic wave, the ultrasonic probe has a contact surface having contact with the fixation section and parallel to the device surface, and the fixation section has a plane along the contact surface.

In the application example with this configuration, the ultrasonic probe has the contact surface parallel to the device surface. Thus, it is possible to smoothly change the relative position between the ultrasonic probe and the fixation section.

Specifically, in the case in which the ultrasonic device is fixed to the housing of the ultrasonic probe provided with the contact surface, and the first fixation section is made movable relatively to the housing, when fixing the relative position of the first fixation section to the second fixation section of the fixation section, and then moving the first fixation section and the ultrasonic device relatively to each other, the ultrasonic device and the housing move relatively to the fixation section. On this occasion, in the application example, since the fixation section has the surface along the contact surface, the contact surface is guided by the plane, and thus, it becomes possible to smoothly displace the housing.

In the ultrasonic probe unit according to the application example, it is preferable that the displacement mechanism displaces the first fixation section.

As the displacement mechanism, it is possible to adopt a configuration of moving the ultrasonic device relatively to the first fixation section. However, there is a possibility that the transmission characteristic and the reception characteristic of the ultrasonic wave vary due to the stress applied to the ultrasonic device. In contrast, by adopting the configuration of moving the first fixation section, the problem described above can be suppressed.

In the ultrasonic probe unit according to the application example, it is preferable that the ultrasonic device has a device surface, which is at least one of a surface adapted to transmit the ultrasonic wave and a surface adapted to receive the ultrasonic wave, and the adjustment section includes a rotary mechanism adapted to rotate at least one of the ultrasonic device and the first fixation section around a rotational axis crossing a normal line of the device surface.

In the application example with this configuration, at least either one of the ultrasonic device and the first fixation section is rotated by the rotary mechanism. On this occasion, for example, the transmission direction of the ultrasonic wave of the ultrasonic device and the reception direction of the ultrasonic wave, in which the ultrasonic wave can be received with high sensitivity, can be rotated around the rotational axis. Therefore, even in the case in which the transmission direction (or the reception direction of the ultrasonic wave) of the ultrasonic wave is shifted from the transmission target (or the transmission (reflection) position of the ultrasonic wave to be received) of the ultrasonic wave in the object, it is possible to easily adjust the angle of the ultrasonic device with respect to the object using the rotary mechanism.

In the ultrasonic probe unit according to the application example, it is preferable that the ultrasonic device has a one-dimensional array structure in which a plurality of ultrasonic transducers is arranged along a first direction parallel to the device surface, and the rotational axis is parallel to the first direction.

In the application example with this configuration, the ultrasonic device has the ultrasonic transducers arranged in the first direction. In such a configuration, similarly to the application example described above, when transmitting the ultrasonic wave, for example, it becomes possible to control the transmission direction of the ultrasonic wave in the scan plane including a straight line along the first direction, in which the ultrasonic transducers are arranged, and perpendicular to the device surface, but the transmission direction of the ultrasonic wave cannot be changed in the second direction crossing the first direction. In contrast, by rotating at least one of the ultrasonic transducer and the first fixation section around the rotational axis parallel to the first direction as in the application example, it is possible to rotate (tilt) the scan plane. Therefore, even in the case in which the transmission direction of the ultrasonic wave and the transmission target of the ultrasonic wave in the object are shifted from each other along the second direction, the shift can easily be corrected by the rotary mechanism.

In the ultrasonic probe unit according to the application example, it is preferable that the rotary mechanism rotates the ultrasonic device.

In the application example with this configuration, by rotating the ultrasonic device, the scan plane is rotated (tilted). In this case, by combining the displacement of the first fixation section relatively to the ultrasonic device along the device surface and the rotation of the ultrasonic device with each other, the transmission/reception direction of the ultrasonic wave of the ultrasonic device can more accurately be adjusted.

In the ultrasonic probe unit according to the application example, it is possible that the rotary mechanism has a configuration of rotating the first fixation section.

In this case, no stress is applied to the ultrasonic device by the rotary mechanism, and the deterioration of the transmission characteristics and the reception characteristics of the ultrasonic wave in the ultrasonic device can be suppressed.

In the ultrasonic probe unit according to the application example described above, it is preferable that the ultrasonic probe has a contact surface having contact with the fixation section, and the contact surface is curved convexly toward a direction from the device surface toward the fixation section when being cut by a plane perpendicular to the rotational axis.

In the application example with this configuration, the ultrasonic probe has the contact surface curved convexly. Therefore, in the case in which the ultrasonic device is fixed to the housing of the ultrasonic probe provided with the contact surface, and the first fixation section is made movable relatively to the housing, when fixing the relative position of the first fixation section to the second fixation section of the fixation section, and then rotating the first fixation section relatively to the ultrasonic device, the ultrasonic device and the housing rotate around the rotational axis. On this occasion, in the application example, since the contact surface is curved convexly, when rotating the housing, there is no chance for a part of the housing to have contact with the fixation section to hinder the rotation, and thus, smooth positioning can be achieved.

In the ultrasonic probe unit according to the application example, it is preferable that the fixation section has a curved surface curved along the contact surface.

In the case in which the contact surface of the housing is curved as described above, by providing the fixation section also with the curved surface curved along the contact surface, it becomes possible to smoothly move the housing with the contact surface guided by the curved surface.

It is preferable that the ultrasonic probe of the application example includes an acoustic lens protruding from a contact surface having contact with the fixation section toward the object.

In the application example with this configuration, the ultrasonic probe is provided with the acoustic lens. Therefore, it is possible to converge the ultrasonic transmitted from the ultrasonic device at a predetermined position in the measurement object, and further, it is possible to uniformly spread the ultrasonic wave reflected at the predetermined position to the entire area of the ultrasonic device. Therefore, it is possible to improve the efficiency of the transmission and reception process of the ultrasonic wave.

In the ultrasonic probe unit according to the application example, it is preferable that the fixation section has a frame body adapted to house the acoustic lens.

In the application example with this configuration, the fixation section has the frame body capable of housing the acoustic lens. In such a configuration, by housing the acoustic lens in the frame body, the acoustic lens does not hinder the adjustment of the relative position between the ultrasonic device and the first fixation section, and the adjustment operation can be performed smoothly. Further, since the frame can be filled with the acoustic matching material such as gel, a drop in output of the ultrasonic wave between the object and the ultrasonic probe can be suppressed.

In the ultrasonic probe unit according to the application example, it is preferable that the first fixation section and the second fixation section are each a magnet.

In the application example with this configuration, it is possible to fix the ultrasonic probe to the fixation section with a simple configuration. Further, since there is adopted the configuration of applying the magnetic force between the first fixation section and the second fixation section to fix the ultrasonic probe to the fixation section, positioning of the ultrasonic probe is easily performed compared to the configuration of, for example, making the click part engage with the engagement part.

An ultrasonic probe according to an application example of the invention is an ultrasonic probe to be fixed to a fixation section fixed to an object, including an ultrasonic device adapted to perform at least one of transmission and reception of an ultrasonic wave, a first fixation section a relative position of which to a second fixation section provided to the fixation section is fixed to thereby fix the ultrasonic probe to the fixation section, and an adjustment section adapted to adjust a relative positional relationship between the ultrasonic device and the first fixation section.

In this application example, similarly to the application example described above, by adjusting the relative positional relationship between the ultrasonic device and the first fixation section using the adjustment section, it is possible to change the relative position of the ultrasonic device to the object. Therefore, even in the case in which the transmission direction (or the reception direction of the ultrasonic wave) of the ultrasonic wave is shifted from the transmission target (or the transmission (reflection) position of the ultrasonic wave to be received) of the ultrasonic wave in the object, it is possible to easily adjust the relative position of the ultrasonic device to the object using the adjustment section, and thus, it is possible to easily fix the ultrasonic probe to the desired position of the object.

An ultrasonic apparatus according to an application example of the invention includes an ultrasonic probe, a fixation section adapted to fix the ultrasonic probe to an object, and a control section adapted to control the ultrasonic probe, the ultrasonic probe includes an ultrasonic device adapted to perform at least one of transmission and reception of an ultrasonic wave, a first fixation section, and an adjustment section adapted to adjust a relative positional relationship between the ultrasonic device and the first fixation section, and the fixation section includes a fixation surface to be fixed to the object, and a second fixation section a relative position of which to the first fixation section is fixed to thereby fix the ultrasonic probe to the object.

In this application example, similarly to the application example described above, by adjusting the relative positional relationship between the ultrasonic device and the first fixation section using the adjustment section, it is possible to easily fix the ultrasonic probe to the desired position of the object. Therefore, in the ultrasonic apparatus, in the case of, for example, measuring the measurement target at a predetermined depth in the object, the measurement result with respect to the measurement target can accurately be obtained.

### BRIEF DESCRIPTION OF THE DRAWINGS

The invention will be described with reference to the accompanying drawings, wherein like numbers reference like elements.

FIG. 1 is a diagram showing a schematic configuration of an ultrasonic apparatus according to a first embodiment of the invention.

FIG. 2 is a schematic cross-sectional view of an ultrasonic probe and a fixation section in the case of fixing the ultrasonic probe to a body surface of a living body in the first embodiment.

FIG. 3 is a plan view showing a general configuration of the fixation section in the first embodiment.

FIG. 4 is a plan view of an element substrate of an ultrasonic device according to the first embodiment viewed from a sealing plate side.

FIG. 5 is a cross-sectional view of the ultrasonic device cut along the line B-B shown in FIG. 4.

FIG. 6 is a schematic cross-sectional view showing a cross-section of the ultrasonic probe according to the first embodiment along the X-Y plane.

FIG. 7 is a diagram schematically showing the condition in which a scan plane of the ultrasonic probe has been changed in the first embodiment.

FIG. 8 is a schematic cross-sectional view of an ultrasonic probe according to a second embodiment and a fixation section in the case of fixing the ultrasonic probe to a body surface of a living body.

FIG. 9 is a schematic cross-sectional view showing a cross-section of the ultrasonic probe according to the second embodiment along the X-Y plane.

FIG. 10 is a diagram schematically showing the condition in which the angle of the scan plane of the ultrasonic probe has been changed in the second embodiment.

FIG. 11 is a schematic cross-sectional view of an ultrasonic probe according to a modified example of the second embodiment and a fixation section in the case of fixing the ultrasonic probe to a body surface of a living body.

FIG. 12 is a schematic cross-sectional view of an ultrasonic probe according to a third embodiment and a fixation section in the case of fixing the ultrasonic probe to a body surface.

FIG. 13 is a schematic cross-sectional view showing a cross-section of the ultrasonic probe according to the third embodiment along the X-Y plane.

FIG. 14 is a schematic cross-sectional view of an ultrasonic probe according to a modified example and a fixation section in the case of fixing the ultrasonic probe to a body surface.

FIG. 15 is a diagram for explaining an outline of a swing mechanism (an adjustment section) of a probe-side magnet in the modified example shown in FIG. 14.

### DESCRIPTION OF EXEMPLARY EMBODIMENTS

### First Embodiment

An ultrasonic apparatus according to the first embodiment will hereinafter be described with reference to the accompanying drawings.

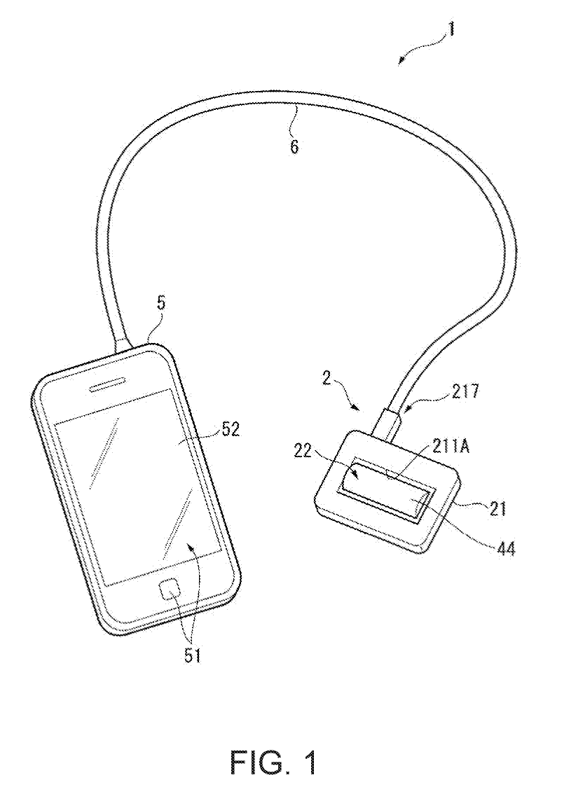

FIG. 1 is a diagram showing a schematic configuration of an ultrasonic apparatus 1 according to the first embodiment.

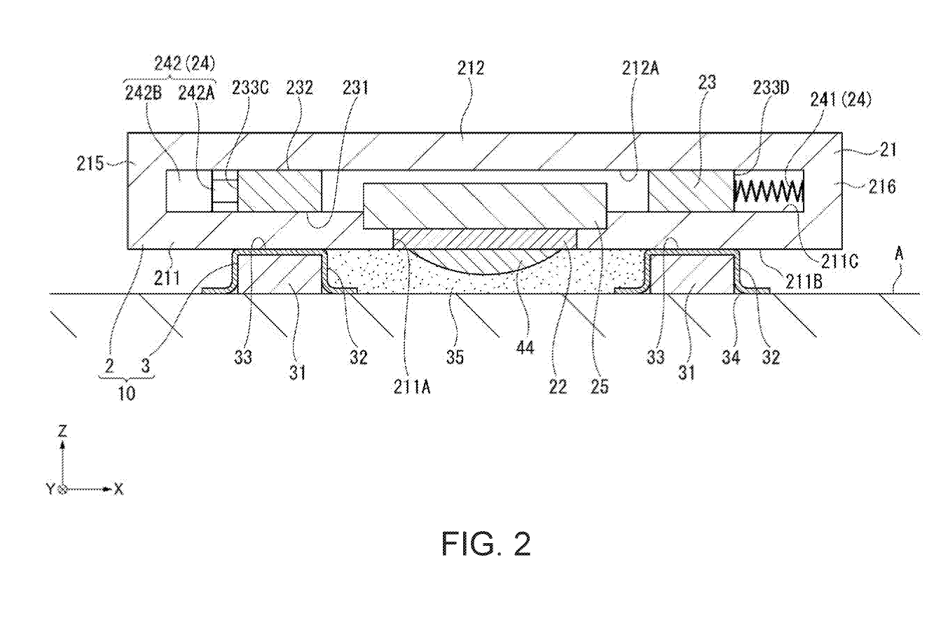

FIG. 2 is a schematic cross-sectional view of an ultrasonic probe 2 and a fixation section 3 in the case of fixing the ultrasonic probe 2 to a body surface of a living body in the first embodiment.

As shown in FIG. 1, the ultrasonic apparatus 1 according to the present embodiment is provided with an ultrasonic probe 2, and a control device 5 (a control section) electrically connected to the ultrasonic probe 2 via, for example, a cable 6.

The ultrasonic apparatus 1 is a device for measuring a shape and a state (e.g., blood flow) of a part (e.g., an organ such as a blood vessel or a liver) in a living body A as an object by fixing the ultrasonic probe 2 to the body surface of the living body A via the fixation section 3 to transmit (perform a transmission process of) an ultrasonic wave, and then receive (perform a reception process of) the ultrasonic wave reflected by the part in the living body A. In other words, the fixation section 3 (see FIG. 2) has a function of fixing the ultrasonic probe 2 to the living body A, and at the same time propagating the ultrasonic wave between the ultrasonic probe 2 and the living body A. Further, in the present embodiment, the ultrasonic probe 2 and the fixation section 3 constitute an ultrasonic probe unit 10.

In such an ultrasonic apparatus 1, in order to perform measurement of a predetermined measurement target part of the living body A, firstly, the fixation section 3 is fixed to the body surface of the living body A, and then the ultrasonic probe 2 is fixed to the fixation section 3. Thus, it is possible to easily fix the ultrasonic probe 2 to the body surface.

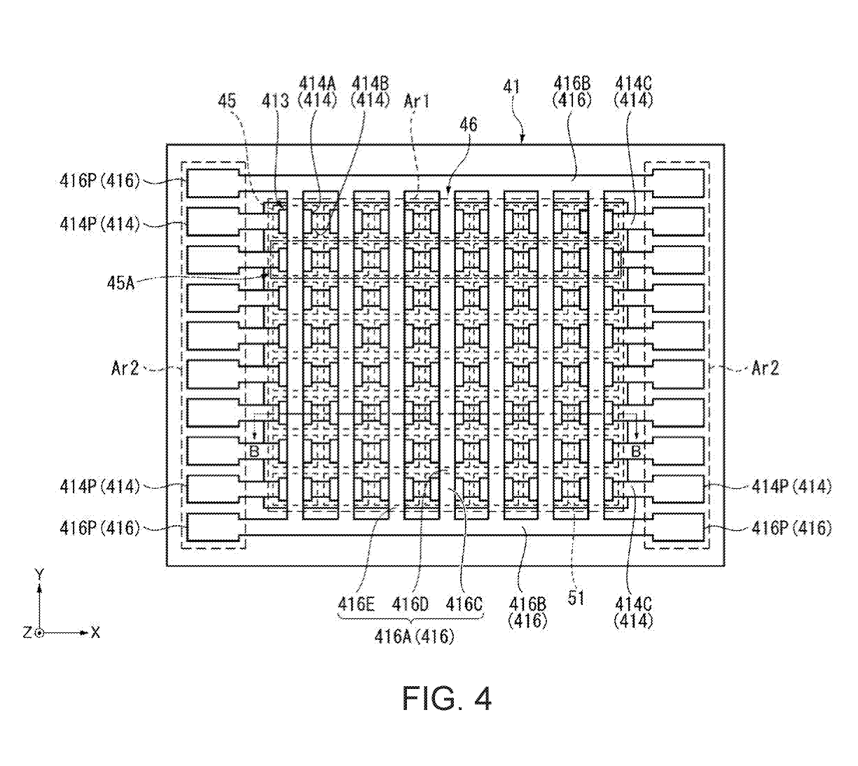

Incidentally, although the detailed configuration will be described later, the ultrasonic probe 2 according to the present embodiment has an ultrasonic device 22 for outputting an ultrasonic wave, and the ultrasonic device 22 has a one-dimensional array structure having a plurality of ultrasonic transducer groups 45A (see FIG. 4) arranged in the Y direction (a first direction, a scanning direction). In such an ultrasonic probe 2, the ultrasonic wave output from the ultrasonic device 22 propagates along a plane (scan plane) passing through the center in the X direction (a second direction, a slicing direction) of each of the ultrasonic transducer groups 45A and perpendicular to the X direction. Therefore, although accurate ultrasonic measurement can be achieved in the case in which the measurement object exists in the scan plane, in the case in which, for example, the measurement object exists at a position distant in the X direction (the slicing direction) from the scan plane, the accurate ultrasonic measurement cannot be achieved in some cases. In contrast, the ultrasonic probe 2 according to the present embodiment is configured so that the position of the ultrasonic device 22 can be adjusted along the X direction with respect to the fixation section 3. Thus, the position of the ultrasonic device 22 can be adjusted so that the measurement object is disposed along the scan plane, and it is possible to perform the accurate ultrasonic measurement.

Hereinafter, the ultrasonic probe unit 10 according to the present embodiment will be described in detail.

### Configuration of Fixation Section 3

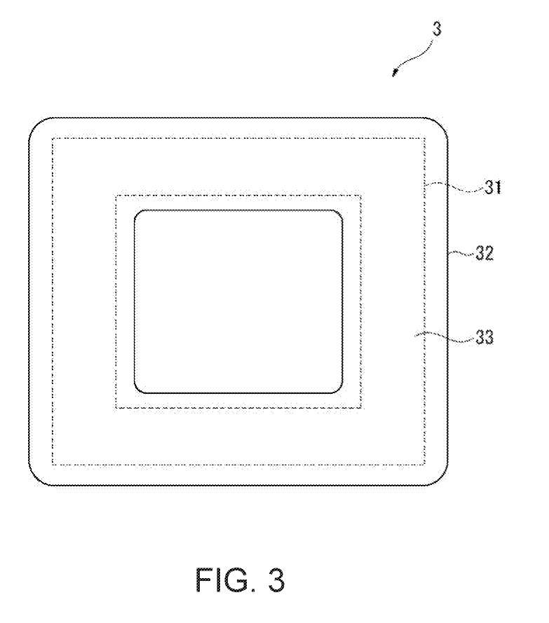

FIG. 3 is a plan view showing a general configuration of the fixation section 3 in the first embodiment.

As shown in FIG. 2 and FIG. 3, the fixation section 3 is provided with a fixation section-side magnet 31 as a second fixation section, and a fixation tape 32 for fixing the fixation section-side magnet 31 to the body surface of the living body A. Further, the fixation section 3 is provided with a probe-opposed surface 33, which is a surface on a +Z side defining the direction perpendicular to the X-Y plane as the Z direction (assuming that the −Z side corresponds to the living body A side), and a fixation surface 34, which is a surface on the −Z side, and is fixed to the body surface of the living body A. In the present embodiment, the probe-opposed surface 33 is formed to have a roughly flat shape, and by fixing the ultrasonic probe 2 to the probe-opposed surface 33 so as to have contact with each other with the fixation surface 34 fixed to the living body, the ultrasonic probe 2 is fixed to the body surface.

Further, the fixation section 3 is shaped like a frame in a planar view viewed from the Z direction, and an acoustic lens 44 described later of the ultrasonic probe 2 can be housed inside the frame. Specifically, in the planar view described above, the fixation section 3 is shaped like a rectangular frame, and an acoustic matching material 35 such as gel is disposed in the opening part (inside the rectangular frame) formed in a central part of the fixation section 3. The acoustic matching material 35 has an equivalent acoustic impedance to that of the body surface, and is capable of preventing a gap between the acoustic lens 44 and the body surface from occurring in the case in which the ultrasonic probe 2 is fixed to the fixation section 3.

The fixation section-side magnet 31 is a frame-like member having a rectangular outer shape in the planar view viewed along the Z direction, and having an opening in the central part. The fixation section-side magnet 31 is preferably configured as, for example, a single bulk member, and can thus suppress deformation of the fixation section-side magnet 31. It should be noted that as the fixation section-side magnet 31, a sheet-like magnet having elasticity can also be used. In this case, it is possible to make the fixation section-side magnet 31 adhere along the body surface, and separation from the body surface and misalignment of the fixation section 3 can be prevented.

The fixation tape 32 has a adhesive surface having an adherence property, and are made to adhere to the body surface in the state of covering the fixation section-side magnet 31 with the adhesive surface side. Thus, the fixation section-side magnet 31 is fixed to the body surface. In other words, a part of the adhesive surface of the fixation tape 32 to be made to adhere to the body surface and a surface of the fixation section-side magnet 31 to be made to have contact with the body surface form the fixation surface 34.

It should be noted that although in the present embodiment, there is illustrated the configuration of fixing the fixation section-side magnet 31 to the body surface with the fixation tape 32, it is also possible to form an adhesive surface in the surface on the −Z side of the fixation section-side magnet to fix the fixation section-side magnet 31 to the body surface. Further, it is also possible to fix the fixation section-side magnet 31 to the body surface using a fixing jig.

### Configuration of Ultrasonic Probe 2

The ultrasonic probe 2 is provided with a housing 21, the ultrasonic device 22 fixed to the inside of the housing 21 and partially exposed to the outside, a probe-side magnet 23 disposed inside the housing 21 so as to be able to move along the X direction relatively to the housing 21, and forming a first fixation section, a displacement mechanism 24 for displacing the probe-side magnet 23 with respect to the housing 21, and a wiring board 25 provided with a driver circuit for controlling the ultrasonic device 22, and so on. In the ultrasonic probe 2, the relative position between the fixation section-side magnet 31 and the probe-side magnet 23 attracting each other with magnetic force is uniquely determined, and thus, the ultrasonic probe 2 is fixed to the fixation section 3. In the present embodiment, the probe-side magnet 23 can be displaced along the X direction with respect to the housing 21 (i.e., the ultrasonic device 22) in the ultrasonic probe 2. Here, even if the probe-side magnet 23 is displaced with respect to the housing 21, the relative position between the probe-side magnet 23 and the fixation section-side magnet 31 does not change. Therefore, the relative position of the ultrasonic device 22 to the fixation section 3 is changed, and thus, it is possible to change the relative position of the ultrasonic device 22 to the body surface.

### Configuration of Ultrasonic Device 22

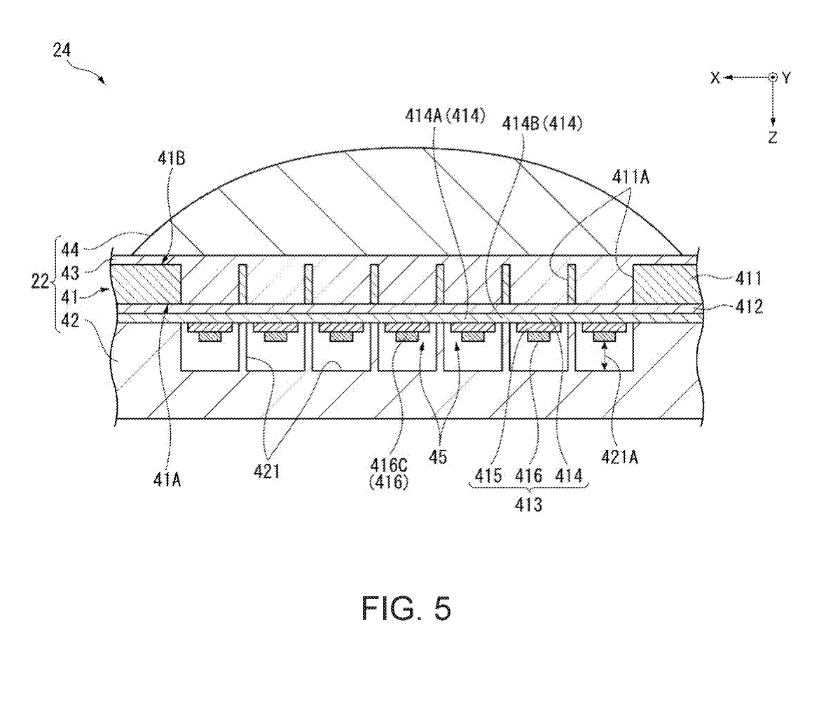

FIG. 4 is a plan view of an element substrate 41 in the ultrasonic device 22 viewed from a sealing plate 42 side. FIG. 5 is a cross-sectional view of the ultrasonic device 22 cut along the line B-B shown in FIG. 4.

As shown in FIG. 5, the ultrasonic device 22 is constituted by the element substrate 41, the sealing plate 42, an acoustic matching layer 43, and the acoustic lens 44.

### Configuration of Element Substrate 41

As shown in FIG. 5, the element substrate 41 is provided with a substrate main body part 411, a vibrating film 412 disposed on the sealing plate 42 side of the substrate main body part 411, and piezoelectric elements 413 stacked on the vibrating film 412. Here, in the following description, a surface of the element substrate 41 opposed to the sealing plate 42 is referred to as a rear surface 41A, and a surface located on the opposite side to the rear surface 41A is referred to as an operating surface 41B (corresponding to a device surface). It should be noted that the normal direction of the operating surface 41B is assumed to roughly coincide with the Z direction. Further, in a planar view of the element substrate 41 viewed from the thickness direction of the substrate, a central area of the element substrate 41 forms an array region Ar1, and in the array region Ar1, a plurality of ultrasonic transducers 45 is arranged in a matrix.

The substrate main body part 411 is a semiconductor substrate made of, for example, Si. Inside the array region Ar1 in the substrate main body part 411, there are disposed aperture parts 411A corresponding respectively to the ultrasonic transducers 45. Further, the aperture parts 411A are closed by the vibrating film 412 disposed on the rear surface 41A side of the substrate main body part 411.

The vibrating film 412 is formed of, for example, SiO₂ or a laminated body of SiO₂ and ZrO₂, and is disposed so as to cover the entire area on the rear surface 41A side of the substrate main body part 411. The thickness dimension of the vibrating film 412 becomes sufficiently small one with respect to that of the substrate main body part 411. In the case of forming the substrate main body part 411 using Si and forming the vibrating film 412 using SiO₂, by performing an oxidation treatment on, for example, the surface on the rear surface 41A side of the substrate main body part 411, it becomes possible to easily form the vibrating film 412 having a desired thickness dimension. Further, in this case, by performing an etching treatment on the substrate main body part 411 using the vibrating film 412 made of SiO₂ as an etching stopper, it becomes possible to easily form the opening parts 411A described above.

Further, as shown in FIG. 5, on the vibrating film 412 closing each of the opening parts 411A, there are disposed the piezoelectric elements 413 each of which is a laminated body of a lower-part electrode 414, a piezoelectric film 415, and an upper-part electrode 416. Here, the vibrating film 412, which closes the aperture part 411A, and the piezoelectric element 413 constitute a single ultrasonic transducer 45.

In such an ultrasonic transducer 45, by applying a rectangular-wave voltage having a predetermined frequency between the lower-part electrode 414 and the upper-part electrode 416, it is possible to vibrate the vibrating film 412 in an opening region of each of the opening parts 411A to transmit the ultrasonic wave. Further, when the vibrating film 412 is vibrated by the ultrasonic wave reflected by the object, an electrical potential difference occurs between an upper part and a lower part of the piezoelectric film 415. Therefore, by detecting the electrical potential difference occurring between the lower-part electrode 414 and the upper-part electrode 416, it becomes possible to detect the ultrasonic wave received.

Further, in the present embodiment, as shown in FIG. 4, the ultrasonic transducer array 46 is configured by arranging a plurality of such ultrasonic transducers 45 as described above in the predetermined array region Ar1 of the element substrate 41 along the X direction (the slicing direction) and the Y direction (the scanning direction) crossing (perpendicular to, in the present embodiment) the X direction.

Here, the lower-part electrode 414 is formed so as to be shaped like a straight line along the X direction. Specifically, the lower-part electrode 414 is constituted by lower-part electrode main bodies 414A, each of which is disposed straddling the plurality of ultrasonic transducers 45 arranged along the X direction, and located between the piezoelectric films 415 and the vibrating film 412, lower-part electrode lines 414B each connecting the lower-part electrode main bodies 414A adjacent to each other, and lower-part terminal electrode lines 414C each extracted to one of terminal regions Ar2 located outside the array region Ar1. Therefore, in the ultrasonic transducers 45 arranged in the X direction, the lower-part electrodes 414 are in the same potential.

Further, the lower-part terminal electrode lines 414C each extend to the terminal region Ar2 located outside the array region Ar1, and respectively constitute first electrode pads 414P in the terminal region Ar2. The first electrode pads 414P are respectively connected to terminal parts provided to the wiring board 25.

On the other hand, as shown in FIG. 4, the upper-part electrode 416 has element electrode parts 416A each disposed straddling the plurality of ultrasonic transducers 45 arranged along the Y direction, and common electrode parts 416B each connecting end parts of the element electrode parts 416A extending in parallel to each other. The element electrode parts 416A each have upper-part electrode main bodies 416C each stacked on the piezoelectric film 415, upper-part electrode lines 416D each connecting the upper-part electrode main bodies 416C adjacent to each other, and upper-part terminal electrodes 416E extending outward along the Y direction from the respective ultrasonic transducers 45 disposed on the both end parts in the Y direction.

The common electrode parts 416B are disposed respectively in a +Y side end part and a −Y side end part of the array region Ar1. The common electrode part 416B located on the +Y side connects the upper-part terminal electrodes 416E to each other, which extend toward the +Y side from the ultrasonic transducers 45 disposed in the +Y side end part out of the plurality of ultrasonic transducers 45 disposed along the Y direction. The common electrode part 416B located in the −Y side end part connects the upper-part terminal electrodes 416E extending toward the −Y side to each other. Therefore, in the ultrasonic transducers 45 located inside the array region Ar1, the upper-part electrodes 416 are in the same potential. Further, the pair of common electrode parts 416B described above are disposed along the X direction, and the end parts are each extracted from the array region Ar1 to the terminal region Ar2. Further, the common electrode parts 416B constitute second electrode pads 416P to be connected to the terminal parts of the wiring board 25 in the terminal region Ar2.

In such an ultrasonic transducer array 46 as described above, there is formed a one-dimensional array structure in which the ultrasonic transducers 45 connected by the lower part electrode 414 to each other and arranged in the X direction constitute one ultrasonic transducer group 45A, and the plurality of ultrasonic transducer groups 45A is arranged along the Y direction.

### Configuration of Sealing Plate 42

The sealing plate 42 is formed to have the same planar shape when viewed from the thickness direction as that of, for example, the element substrate 41, and is formed of a semiconductor substrate made of Si or the like, or an insulator substrate. It should be noted that the material and the thickness of the sealing plate 42 affect the frequency characteristics of the ultrasonic transducer 45, and are therefore preferably set based on the central frequency of the ultrasonic wave transmitted/received by the ultrasonic transducer 45.

Further, the sealing plate 42 is provided with a plurality of concave grooves 421, which correspond respectively to the opening parts 411A of the element substrate 41, formed in an array-opposed area opposed to the array region Ar1 of the element substrate 41.

Thus, it results that a gap 421A having a predetermined dimension is provided between the element substrate 41 and the area (inside the opening part 411A) vibrated by the ultrasonic transducer 45 in the vibrating film 412, and the vibration of the vibrating film 412 is prevented from being hindered. Further, it is possible to suppress the problem (cross talk) that the back wave from one ultrasonic transducer 45 enters another ultrasonic transducer 45 adjacent to that ultrasonic transducer 45.

Further, when the vibrating film 412 vibrates, an ultrasonic wave is also emitted toward the sealing plate 42 side (the rear surface 41A side) as the back wave in addition to the aperture part 411A side (the operating surface 41B side). The back wave is reflected by the sealing plate 42, and is then emitted again toward the vibrating film 412 side via the gap 421A. On this occasion, if the phase of the reflected back wave and the phase of the ultrasonic wave emitted from the vibrating film 412 toward the operating surface 41B side are shifted from each other, the ultrasonic wave is attenuated. Therefore, in the present embodiment, the groove depth of each of the concave grooves 421 is set so that the acoustic distance in the gap 421A becomes an odd multiple of a quarter (λ/4) of the wavelength λ of the ultrasonic wave. In other words, the thickness dimensions of the variety of parts of the element substrate 41 and the sealing plate 42 are set taking the wavelength λ of the ultrasonic wave emitted from the ultrasonic transducers 45 into consideration.

Further, it is also possible for the sealing plate 42 to have a configuration in which, for example, opening parts (not shown) are disposed at positions opposed to the terminal regions Ar2 of the element substrate 41 so as to correspond to the respective electrode pads 414P, 416P disposed in the terminal regions Ar2. On this occasion, by providing through electrodes (TSV; through-silicon via) penetrating the sealing plate 42 in the thickness direction to the respective opening parts, the electrode pads 414P, 416P are connected to the terminal parts in the wiring board 25 via the through electrodes, respectively. Further, it is also possible to adopt a configuration in which, for example, FPC (flexible printed circuits), cable lines, wires, or the like are inserted into the opening parts to connect the electrode pads 414P, 416P and the wiring board 25 to each other.

### Configuration of Acoustic Matching Layer 43 and Acoustic Lens 44

As shown in FIG. 5, the acoustic matching layer 43 is disposed on the operating surface 41B side of the element substrate 41. Specifically, the acoustic matching layer 43 is formed so as to fill in the aperture parts 411A of the element substrate 41, and to have a predetermined thickness dimension from the operating surface 41B side of the substrate main body part 411.

The acoustic lens 44 is disposed on the acoustic matching layer 43, and is exposed to the outside from the sensor window 211A of the housing 21 as shown in FIG. 1 and FIG. 2. As shown in FIG. 5, the acoustic lens 44 has a cylindrical shape the surface shape of which is an arc-like shape in a cross-sectional view along the X direction (the slicing direction).

It should be noted that the acoustic matching layer and the acoustic lens 44 efficiently propagate the ultrasonic wave emitted from the ultrasonic transducers 45 to the living body A as the measurement object, and further propagate the ultrasonic wave, which has been reflected in the living body A, to the ultrasonic transducers 45 with efficiency. Therefore, the acoustic matching layer 43 and the acoustic lens 44 are set to have an acoustic impedance intermediate between the acoustic impedance of the ultrasonic transducers 45 of the element substrate 41 and the acoustic impedance of the living body. As a material having such acoustic impedance as described above, there can be cited, for example, silicone.

### Configuration of Wiring Board 25

The wiring board 25 is a substrate to which the ultrasonic device 22 is fixed, and is provided with the terminal parts electrically connected to the respective electrode pads 414P, 416P. Further, the wiring board 25 is provided with a driver circuit for driving the ultrasonic device 22, and so on. Specifically, the wiring board 25 is provided with a transmission circuit for transmitting the ultrasonic wave from the ultrasonic transducer array 46, a reception circuit for processing the received signal in the case in which the ultrasonic wave has been received by the ultrasonic transducer array 46, and so on. Further, the wiring board 25 is connected to the control device 5 via the cable 6 or the like, and drives the ultrasonic device 22 based on an instruction from the control device 5.

The ultrasonic device 22 and the wiring board 25 constitute the ultrasonic sensor.

### Configuration of Housing 21

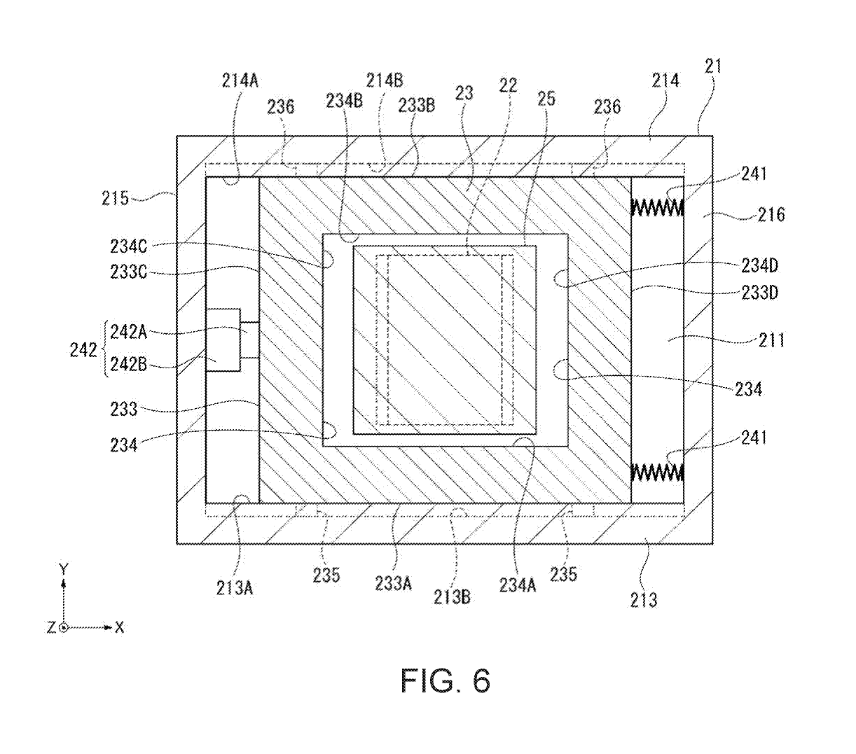

FIG. 6 is a schematic cross-sectional view showing a cross-section of the ultrasonic probe 2 along the X-Y plane.

The housing 21 is formed so as to be shaped like a box having a roughly rectangular planar shape as shown in FIG. 1, and is provided with a bottom surface part 211 on the −Z side, a top surface part 212 on the +Z side, a first side surface part 213 on the −Y side, a second side surface part 214 on the +Y side, a third side surface part 215 on the −X side, and a fourth side surface part 216 of the +X side as shown in FIG. 2 or FIG. 6. In an internal space formed by these parts 211 through 216, there are housed the ultrasonic device 22, the probe-side magnet 23, and the displacement mechanism 24.

As shown in FIG. 2, the bottom surface part 211 has a sensor window 211A located on the −Z side of the housing 21 and exposing a part (the acoustic lens 44) of the ultrasonic device 22, a contact surface 211B as a surface located on the −Z side, and an inner bottom surface 211C as a surface located on the +Z side, and the ultrasonic device 22 is fixed to the bottom surface part 211. The contact surface 211B is a surface having contact with the probe-opposed surface 33 of the fixation section 3, and is formed to have a roughly flat shape. The inner bottom surface 211C is a surface having contact with a lower surface 231 of the probe-side magnet 23 described later, and is formed to have a roughly flat shape. It should be noted that the ultrasonic device 22 is fixed to a position overlapping the sensor window 211A.

The top surface part 212 is located on the +Z side of the housing 21, and has a top surface 212A formed to have a roughly flat shape. The top surface 212A has contact with an upper surface 232 of the probe-side magnet 23 described later.

The distance in the Z direction between the bottom surface part 211 and the top surface part 212 (i.e., the distance between the inner bottom surface 211C and the top surface 212A) is roughly the same as or slightly longer than the thickness dimension in the Z direction of the probe-side magnet 23. Thus, it is possible for the probe-side magnet 23 to smoothly move along the inner bottom surface 211C and the top surface 212A.

The first side surface part 213 is a wall part, which is located on the −Y side of the bottom surface part 211 (the top surface part 212), and is parallel to the X-Z plane. The first side surface part 213 has a first inner surface 213A and a first groove part 213B. The first inner surface 213A is a surface having contact with a first outer side surface 233A of the probe-side magnet 23 described later, and is formed to have a roughly flat shape. The first groove part 213B is a groove part disposed along the X direction so as to be recessed in the −Y direction from the first inner surface 213A side. Into the first groove part 213B, there are inserted first protruding parts 235 of the probe-side magnet 23 described later.

The second side surface part 214 is a wall part configured so as to be mirror symmetric about the X-Z plane with the first side surface part 213. In other words, the second side surface part 214 has a second inner surface 214A having contact with a second outer side surface 233B of the probe-side magnet 23, and a second groove part 214B into which second protruding parts 236 of the probe-side magnet 23 are inserted.

The distance in the Y direction between the first side surface part 213 and the second side surface part 214 (i.e., the distance between the first inner surface 213A and the second inner surface 214A) is roughly the same as or slightly longer than the thickness dimension in the Y direction of the probe-side magnet 23. Thus, it is possible for the probe-side magnet 23 to smoothly move along the first inner surface 213A and the second inner surface 214A. In other words, the bottom surface part 211, the top surface part 212, the first side surface part 213, and the second side surface part 214 function as a guide for the probe-side magnet 23.

The third side surface part 215 is a wall part, which is located on the −X side of the bottom surface part 211 (the top surface part 212), and is parallel to the Y-Z plane. The fourth side surface part 216 is a wall part, which is located on the +X side of the bottom surface part 211 (the top surface part 212), and is parallel to the Y-Z plane.

The distance between the third side surface part 215 and the fourth side surface part 216 is longer than the dimension in the X direction of the probe-side magnet 23. In other words, spaces are formed between a surface (a third outer side surface) 233C on the −X side of the probe-side magnet 23 and the inner surface of the third side surface part 215, and between a surface (a fourth outer side surface) 233D on the +X side of the probe-side magnet 23, respectively. Thus, the spaces allowing the probe-side magnet 23 to move forward and backward along the X direction are formed inside the housing 21.

Further, in a part (e.g., a side surface in the example shown in FIG. 1) of the housing 21, there is disposed an insertion hole 217 through which the cable 6 for connecting the ultrasonic probe 2 and the control device 5 to each other so as to be able to communicate with each other is inserted. The gap between the cable 6 and the insertion hole 217 is filled with, for example, a resin material to thereby ensure a waterproof property. It should be noted that although in the present embodiment, there is shown a configuration example in which the ultrasonic probe 2 and the control device 5 are connected to each other using the cable 6, the configuration is not limited to this example, and it is also possible to, for example, connect the ultrasonic probe 2 and the control device 5 to each other with wireless communication, or dispose a variety of constituents of the control device 5 inside the ultrasonic probe 2.

### Configuration of Probe-Side Magnet 23

The probe-side magnet 23 shown in FIG. 2 and FIG. 6 is a frame-like member having a rectangular outer shape in the planar view viewed along the Z direction, and having an opening in the central part. In the present embodiment, the probe-side magnet 23 has roughly the same outer shape as that of the fixation section-side magnet 31 in the planar view viewed along the Z direction.

The probe-side magnet 23 is formed as, for example, a single bulk member, and has the rigidity with which a deformation due to the stress can be suppressed.

The probe-side magnet 23 is provided with the lower surface 231 (see FIG. 2) on the −Z side, the upper surface 232 on the +Z side, the outer side surfaces 233, the inner side surfaces 234, the first protruding parts 235, and the second protruding parts 236.

The lower surface 231 is a surface having contact with the bottom surface part 211 of the housing 21, and the upper surface 232 is a surface having contact with the top surface part 212.

The outer side surfaces 233 includes the first outer side surface 233A on the −Y side, the second outer side surface 233B on the +Y side, the third outer side surface 233C on the −X side, and the fourth outer side surface 233D.

The first outer side surface 233A has contact with the first inner surface 213A of the housing 21. The first outer side surface 233A is provided with the first protruding parts 235 protruding in the −Y direction, and inserted into the first groove part 213B. The second outer side surface 233B has contact with the second inner surface 214A of the housing 21. The second outer side surface 233B is provided with the second protruding parts 236 protruding in the +Y direction, and inserted into the second groove part 214B. The third outer side surface 233C is opposed to, and separated in the X direction from the third side surface part 215 of the housing 21. The fourth outer side surface 233D is opposed to, and separated in the X direction from the fourth side surface part 216.

The inner side surfaces 234 includes the first inner side surface 234A located on the −Y side and parallel to the X-Z plane, the second inner side surface 234B opposed to the first inner side surface 243A, the third inner side surface 234C located on the −X side, and crossing the first inner side surface 234A and the second inner side surface 234B, and the fourth inner side surface 234D opposed to the third inner side surface 234C. The ultrasonic device 22 is disposed at the position surrounded by these inner side surfaces 234A through 234D.

Here, the distance dimension between the third inner side surface 234C and the fourth inner side surface 234D is larger than the dimension in the X direction of the ultrasonic device 22. Thus, the spaces allowing the probe-side magnet 23 to move forward and backward inside the housing 21 along the X direction are formed between the housing 21 and the probe-side magnet 23 in the X direction, and between the ultrasonic device 22 and the probe-side magnet 23, respectively. Further, it is preferable for the distance (a sum of the distance from the ultrasonic device 22 to the third inner side surface 234C, and the distance from the ultrasonic device 22 to the fourth inner side surface 234D) between the ultrasonic device 22 and the probe-side magnet 23 in the X direction to be equal to or longer than the distance which the probe-side magnet 23 can move in the X direction. Thus, as described above, even in the case in which the probe-side magnet 23 moves along the X direction, the probe-side magnet 23 and the ultrasonic device 22 do not interfere with each other.

The probe-side magnet 23 is formed so that the relative position to the fixation section-side magnet 31 described above has a predetermined positional relationship. Specifically, in the present embodiment, the surface (on the living body A side) on the −Z side of the probe-side magnet 23 and the surface (on the ultrasonic probe 2 side) on the +Z side of the fixation section-side magnet 31 have magnetic poles different from each other, and attract each other to thereby fix the ultrasonic probe 2 to the fixation section 3 at a position where the probe-side magnet 23 and the fixation section-side magnet 31 overlap each other in the planar view viewed from the Z direction.

It should be noted that it is possible for the probe-side magnet 23 and the fixation section-side magnet 31 to have the magnetic poles different between the surface on the −Z side and the surface on the +Z side, or the magnetic poles different in the X-Y plane.

In the former case, there can be illustrated a configuration in which the surface on the −Z side of the probe-side magnet 23 has the south pole, and the surface on the +Z side of the fixation section-side magnet 31 has the north pole. In this case, the fixation direction of the ultrasonic probe 2 with respect to the fixation section 3 can be changed as much as an angle corresponding to the planar shapes viewed from the Z direction of the probe-side magnet 23 and the fixation section-side magnet 31. For example, in the case in which the frame shapes of the probe-side magnet 23 and the fixation section-side magnet 31 are each a square shape, it is also possible to rotate the ultrasonic probe 2 as much as 90°, 180°, or 270° in the X-Y plane with respect to the fixation section 3 and then fix the ultrasonic probe 2 to the fixation section 3. Further, in the case in which the frame shapes of the probe-side magnet 23 and the fixation section-side magnet 31 are each a rectangular shape, it is possible to rotate the ultrasonic probe 2 as much as 180° in the X-Y plane with respect to the fixation section 3 and then fix the ultrasonic probe 2 to the fixation section 3.

On the other hand, in the latter case, there can be illustrated the configuration in which, for example, the +X side of the probe-side magnet 23 is the north pole, and the −X side is the south pole, and the +X side of the fixation section-side magnet 31 is the south pole, and the −X side is the north pole. In this case, the fixation direction of the ultrasonic probe 2 with respect to the fixation section 3 can uniquely be determined. Therefore, even in the case in which the ultrasonic probe 2 is slightly shifted or rotated in the X-Y plane with respect to the fixation section 3, the magnetic force acts in the direction of achieving a balanced state, and thus it is possible to correct the position to the position where the probe-side magnet 23 and the fixation section-side magnet 31 overlap each other in the planar view.

### Configuration of Displacement Mechanism 24

The displacement mechanism 24 is an adjustment section, and is provided with a biasing section 241 for biasing the probe-side magnet 23 toward the −X direction, and a pressing section 242 for pressing the probe-side magnet 23 toward the +X direction, and moves the probe-side magnet 23 forward and backward along the X direction relatively to the housing 21.

The biasing section 241 is, for example, a coil spring arranged so as to expand and contract along the X direction, and the end part on the +X side of the biasing section 241 is fixed to the fourth side surface part 216 of the housing 21, and the end part on the −X side of the biasing section 241 has contact with the fourth outer side surface 233D of the probe-side magnet 23. In the example shown in the drawings, the probe-side magnet 23 is biased toward the −X direction with two coil springs. It should be noted that as the biasing section 241, there can be used a leaf spring, a torsion coil spring, an elastic member such as rubber, and so on besides the coil spring.

The pressing section 242 has a pressing part 242A having contact with the third outer side surface 233C on the −X side of the probe-side magnet 23, and pressing the probe-side magnet 23 toward the +X direction, which is an opposite direction to the biasing direction of the biasing section 241, and a drive section 242B for moving the pressing part 242A forward and backward along the X direction. The drive section 242B is provided with, for example, a micrometer head for converting the rotational force around the axis into the forward/backward drive force in the axial direction of a spindle (the pressing part 242A), and a stepping motor for applying the rotational force to the micrometer head, and controls the micrometer head based on the control by the control device 5 to move the pressing part 242A forward and backward. It should be noted that as the drive section 242B, there can be used a variety of types of actuators for moving the pressing part 242A forward and backward along the X direction. Further, as the pressing section 242, it is also possible to adopt a configuration of manually moving the pressing part 242A forward and backward. Specifically, as the pressing section 242, it is also possible to adopt a configuration provided with the micrometer head fixed to the outside of the third side surface part 215 of the housing 21 and a spindle inserted into a through hole provided to the third side surface part 215, and moved forward and backward along the X direction in accordance with the operation of the micrometer head.

### Configuration of Control Device 5

As shown in FIG. 1, the control section 5 is provided with, for example, an operating section 51 and a display section 52. Further, although not shown in the drawings, the control device 5 is provided with a storage section formed of a memory and so on, and an arithmetic section constituted by a central processing unit (CPU) and so on. Further, the control device 5 reads and then executes a variety of programs stored in the storage section with the arithmetic section to thereby output an instruction for controlling drive of the ultrasonic device 22, form an image of the internal structure of the living body A based on the received signal input from the ultrasonic device 22 to make the display section 52 display the image, and measure the biological information such as the blood flow to make the display section 52 display the result, for example. Further, the control device 5 controls the drive of the drive section 242B of the displacement mechanism 24 to move the pressing part 242A forward or backward. As such a control device 5 as described above, there can be used a terminal device such as a tablet terminal, a smartphone, or a personal computer, and a dedicated terminal device for operating the ultrasonic probe 2 can also be used.

### Method of Utilizing Ultrasonic Probe Unit 10

### Fixation of Ultrasonic Probe 2 Using Fixation Section 3

In order to fix the ultrasonic probe 2 to the body surface of the living body A using the fixation section 3, firstly, the fixation section 3 is made to adhere to the body surface. Then, the opening part (within the frame) formed in the center of the fixation section 3 is filled with the acoustic matching material 35.

Then, the ultrasonic probe 2 is disposed on the fixation section 3 in a state in which the bottom part 211 is made to face to the body surface side. Here, due to the magnetic force of the probe-side magnet 23 and the fixation section-side magnet 31, the ultrasonic probe 2 is aligned and then fixed to the fixation section 3 so that the probe-side magnet 23 and the fixation section-side magnet 31 have a positional relationship of being parallel to each other and overlapping each other in the planar view viewed from the Z direction as described above. In such a manner, the ultrasonic probe 2 is fixed to the body surface with the fixation section 3 (see FIG. 2). Further, it results that the space formed by the opening part (within the frame) of the fixation section 3, the ultrasonic probe 2, and the body surface is filled with the acoustic matching material 35, and thus, the bubbles are prevented from occurring between the ultrasonic probe 2 and the living body A. Specifically, in the ultrasonic probe unit 10 according to the present embodiment, even in the case of fixing the ultrasonic probe 2 to the living body A for a long period of time, the problem that the acoustic matching material 35 flows out from the space between the ultrasonic probe and the living body A is suppressed, and thus, it is possible to eliminate or ease the cumbersome operation such as refilling the space with the acoustic matching material 35 during the measurement.

### Positioning of Scan Plane of Ultrasonic Probe 2

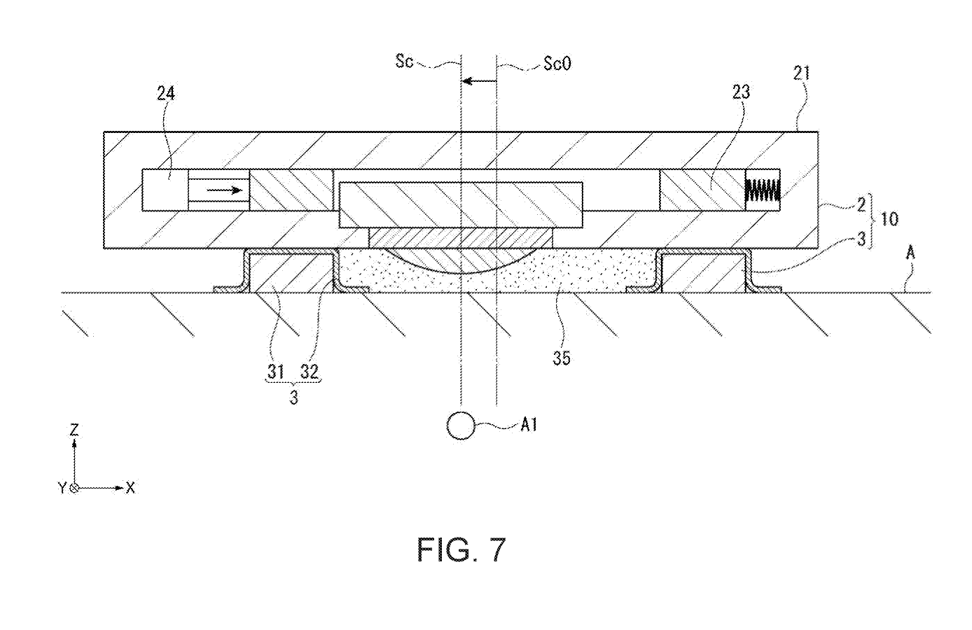

FIG. 7 is a diagram schematically showing the condition in which the scan plane Sc of the ultrasonic probe 2 has been changed.

In the ultrasonic apparatus 1 according to the present embodiment, after fixing the ultrasonic probe 2 to the fixation section 3, the scan plane Sc can be adjusted along the X direction. Specifically, it is possible to drive the displacement mechanism 24 based on the control by the control device 5 to adjust the relative position of the probe-side magnet 23 in the X direction to the housing 21 (the ultrasonic device 22). For example, in the case of displacing the probe-side magnet 23 in the +X direction as shown in FIG. 7, a predetermined positional relationship between the probe-side magnet 23 and the fixation section-side magnet 31 is restored irrespective of the positions of the housing 21 and the probe-side magnet 23 as described above. Specifically, the relative position of the probe-side magnet 23 to the fixation section-side magnet 31 does not change since the fixation section-side magnet 31 and the probe-side magnet 23 attract each other with the magnetic force. Therefore, the housing 21 and the ultrasonic device 22 fixed to the housing 21 are displaced toward the −X direction relatively to the fixation section 3. Therefore, the scan plane Sc is displaced toward the −X direction relatively to the scan plane Sc0 corresponding to the probe-side magnet 23 which has not yet moved. Thus, it is possible to adjust the position of the scan plane Sc so that the measurement object A1 is located on the scan plane Sc. Similarly, by displacing the probe-side magnet 23 toward the −X direction, the position of the scan plane Sc can be moved toward the +X direction.

### Functions and Advantages of First Embodiment

The ultrasonic probe unit 10 is provided with the fixation section 3 and the ultrasonic probe 2. By fixing the probe-opposed surface 33 to the object, the fixation section 3 is fixed to the object.

In the ultrasonic probe 2, by fixing the relative position between the probe-side magnet 23 provided to the ultrasonic probe 2 and the fixation section-side magnet 31 provided to the fixation section 3, the ultrasonic probe 2 is fixed to the object. Further, in the present embodiment, the ultrasonic probe 2 is provided with the displacement mechanism 24 for displacing the probe-side magnet 23 along the X direction relatively to the ultrasonic device 22 to thereby adjust the relative positional relationship between the ultrasonic device 22 and the probe-side magnet 23. As described above, when fixing the ultrasonic probe 2 to the object, the relative position between the probe-side magnet 23 and the fixation section-side magnet 31 is fixed. Therefore, if the relative positional relationship between the ultrasonic device 22 and the probe-side magnet 23 is adjusted by the displacement mechanism 24, the relative position of the ultrasonic device 22 to the object changes.

Therefore, even in the case in which the transmission direction (or the reception direction of the ultrasonic wave) of the ultrasonic wave is shifted from the transmission target (or the transmission (reflection) position of the ultrasonic wave to be received) of the ultrasonic wave in the object when fixing the fixation section 3 to the object, and fixing the ultrasonic probe 2 to the fixation section 3, it is easy to adjust the position of the ultrasonic device 22 so as to be the appropriate position for the object using the displacement mechanism 24. Therefore, in the present embodiment, it is easy to fix the ultrasonic probe 2 to the desired position for the object.

The displacement mechanism 24 displaces the ultrasonic device 22 and the probe-side magnet 23 in a plane parallel to the operating surface 41B along the X direction (slicing direction). Therefore, even in the case in which the positional relationship between the ultrasonic device 22 and the probe-side magnet 23 is adjusted by the displacement mechanism 24, the distance between the ultrasonic device and the object is not changed. Therefore, the distance (the depth) between the ultrasonic device 22 and the transmission target (or the transmission (reflection) position of the ultrasonic wave to be received) of the ultrasonic wave in the object is not changed, and the transmission process and the reception process of the ultrasonic wave high in accuracy can be performed.

Further, in the present embodiment, the ultrasonic device 22 has the one-dimensional array structure in which the ultrasonic transducer groups 45A are arranged in the Y direction (the scanning direction), and is capable of controlling the transmission direction of the ultrasonic wave along the scan plane Sc parallel to the Y-Z plane and perpendicular to the operating surface 41B in accordance with the drive timings of the ultrasonic transducer groups 45A. In such an ultrasonic device 22, it is not achievable to change the transmission direction of the ultrasonic wave in the X direction. Therefore, in the ultrasonic device 22 having the one-dimensional array structure, in the case in which the transmission direction of the ultrasonic wave, namely the scan plane Sc, and the transmission target of the ultrasonic wave in the object are shifted from each other along the X direction, it becomes difficult to correct the shift. In contrast, according to the present embodiment, it is possible to relatively move the probe-side magnet 23 and the ultrasonic device 22 along the X direction using the displacement mechanism 24 to thereby move the scan plane Sc along the X direction. Therefore, as described above, even in the case in which the transmission direction of the ultrasonic wave and the transmission target of the ultrasonic wave in the object are shifted from each other along the X direction, the shift can easily be corrected by the displacement mechanism 24.

The ultrasonic probe 2 has the contact surface 211B parallel to the operating surface 41B. Further, the fixation section 3 has the flat probe-opposed surface 33. Thus, it is possible to move the probe-side magnet 23 relatively to the ultrasonic device 22 along the X direction to smoothly move the ultrasonic probe 2 relatively to the fixation section 3 while being guided by the probe-opposed surface 33 with respect to the fixation section 3.

Here, as the displacement mechanism 24, it is possible to adopt a configuration of moving the ultrasonic device 22 relatively to the probe-side magnet 23. However, there is a possibility that the transmission characteristic and the reception characteristic of the ultrasonic wave vary due to, for example, the stress applied to the ultrasonic device 22 during the movement. In contrast, in the present embodiment, since the displacement mechanism 24 moves the probe-side magnet relatively to the ultrasonic device 22, the problem described above can be prevented.

The ultrasonic device 22 is provided with the acoustic lens 44. Therefore, it is possible to converge the ultrasonic transmitted from the ultrasonic device 22 at a predetermined position in the measurement object, and further, it is possible to uniformly spread the ultrasonic wave reflected at the predetermined position to the entire area of the ultrasonic device. Therefore, it is possible to improve the efficiency of the transmission and reception process of the ultrasonic wave.

The fixation section 3 has the fixation section-side magnet 31 having the frame-like shape, and is capable of housing the acoustic lens 44. As described above, by housing the acoustic lens 44 in the fixation section 3 having the frame-like shape, the acoustic lens 44 does not hinder the adjustment of the relative position between the ultrasonic device 22 and the probe-side magnet 23, and the adjustment operation can be performed smoothly. Further, since the frame can be filled with the acoustic matching material 35 such as gel, a drop in output of the ultrasonic wave between the object and the ultrasonic probe 2 can be suppressed.

Further, in the ultrasonic probe unit 10, due to the magnetic force generated between the probe-side magnet 23 and the fixation section-side magnet 31, the ultrasonic probe 2 is fixed to the fixation section 3. As described above, the ultrasonic probe 2 can be fixed with the simple configuration. Further, it is easy to perform the positioning of the ultrasonic probe 2 compared to the configuration of, for example, making a click part engage with an engaging part. Specifically, in the configuration of making the click part engage with the engaging part, there occurs the work of fitting the click part into the engaging part by an operator. However, in the present embodiment, since the relative position between the fixation section-side magnet 31 and the probe-side magnet 23 is automatically determined by the attractive force due to the magnetic force, the fitting work described above becomes unnecessary.

### Second Embodiment

Then, a second embodiment will be described.

In the first embodiment described above, the ultrasonic probe 2 is configured so that the scan plane to the body surface can be displaced along the X direction by displacing the probe-side magnet 23 along the X direction relatively to the housing 21. In contrast, in the second embodiment, there is adopted a configuration in which the angle of the scan plane to the body surface can be changed by rotating the probe-side magnet 23 with respect to the housing 21. The second embodiment is different in this point from the first embodiment described above.

Hereinafter, an ultrasonic device according to the present embodiment will be described. It should be noted that in the following explanation, the constituents substantially the same as those of the first embodiment are denoted by the same reference symbols, and the explanation thereof will be omitted or simplified.

### Configuration of Ultrasonic Probe 2A

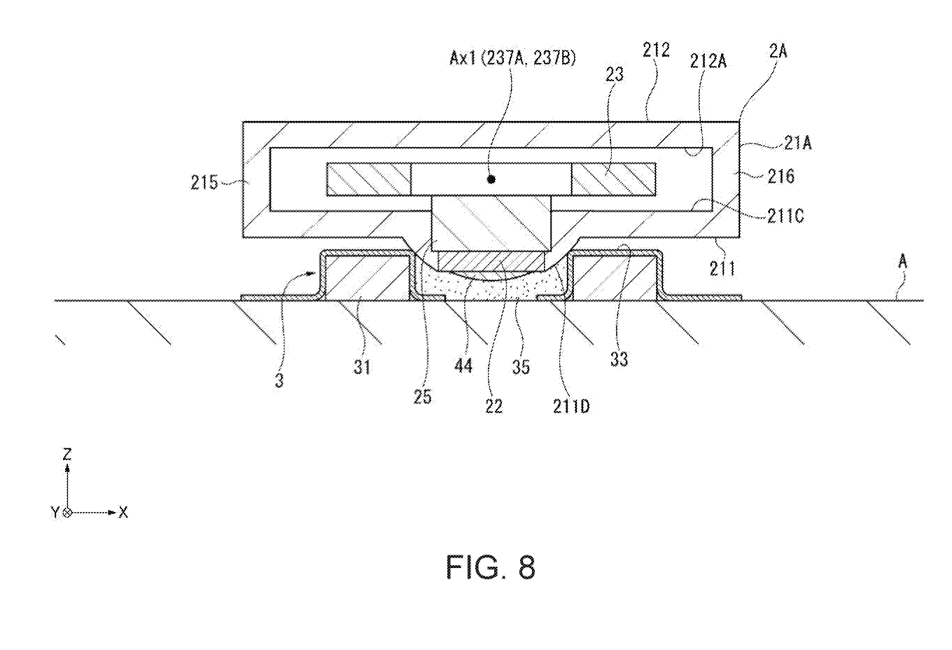

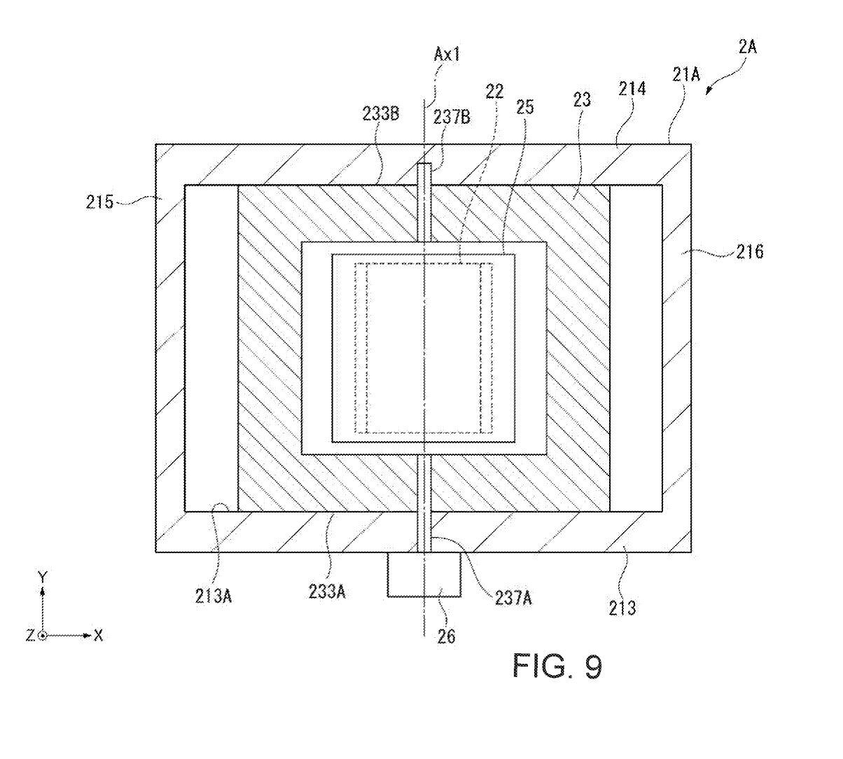

FIG. 8 is a schematic cross-sectional view of the ultrasonic probe 2A according to the second embodiment and the fixation section 3 in the case of fixing the ultrasonic probe 2A to the body surface of the living body. FIG. 9 is a schematic cross-sectional view showing a cross-section of the ultrasonic probe 2A along the X-Y plane.

The ultrasonic probe 2A is provided with a housing 21A, the ultrasonic device 22 fixed to the inside of the housing 21A, a probe-side magnet 23 rotatably disposed inside the housing 21A, a rotary mechanism 26 for rotating the probe-side magnet 23 with respect to the housing 21A, and a wiring board provided with a driver circuit for controlling the ultrasonic device 22, and so on.

The probe-side magnet 23 is supported by the housing 21 with rotary shafts 237A, 237B (see FIG. 9) so as to be rotatable around the center axis (a shaft center Ax1) parallel to the Y direction. Specifically, as shown in FIG. 9, the rotary shaft 237A is pivoted by the first side surface part 213 of the housing 21, and the rotary shaft 237B is concentric with the rotary shaft 237A, and is pivoted by the second side surface part 214. It should be noted that although in the present embodiment, there is shown an example in which the probe-side magnet 23 is supported by the rotary shafts 237A, 237B, but the invention is not limited to this example. For example, in the case in which the ultrasonic device 22 is not located at the rotational center (the shaft center Ax1), it is also possible to adopt a configuration in which the probe-side magnet 23 is supported by a single rotary shaft penetrating the probe-side magnet 23 in the Y direction.

The rotary mechanism 26 is connected to the rotary shaft 237A, and rotates the rotary shaft 237A to thereby rotate the probe-side magnet 23 relatively to the housing 21. As the rotary mechanism 26, there can be used a stepping motor, the rotational amount of which can be controlled based on the control by the control device 5. It should be noted that as the rotary mechanism 26, besides the use of the stepping motor, it is also possible to adopt a configuration in which an operating section is disposed at an end part on the −Y side of the rotary shaft 237A, and the operating section is manually rotated to thereby rotate the probe-side magnet 23.

As shown in FIG. 8, in the present embodiment, the distance dimension between the inner bottom surface 211C and the top surface 212A of the housing 21 is larger than the thickness dimension in the Z direction of the probe-side magnet 23. Thus, there are formed gaps, which enable the rotation of the probe-side magnet 23 around the shaft center Ax1 of the rotary shafts 237A, 237B, between the lower surface 231 of the probe-side magnet 23 and the inner bottom surface 211C, and between the upper surface 232 of the probe-side magnet 23 and the top surface 212A.

Further, in the bottom surface part 211 of the housing 21, a predetermined area centered on the part where the acoustic lens 44 of the ultrasonic device 22 is exposed forms a contact surface 211D curved convexly in a direction from the device surface of the ultrasonic device 22 toward the fixation section 3, namely toward the −Z direction. The contact surface 211D is formed to have a cylindrical shape, which forms a circular arc with the shaft center Ax1 of the rotary shafts 237A, 237B as the central axis in the Z-X plane shown in FIG. 8. Here, the center of the circular arc of the contact surface 211D coincides with the shaft center Ax1.

### Angle Adjustment of Scan Plane of Ultrasonic Probe 2A

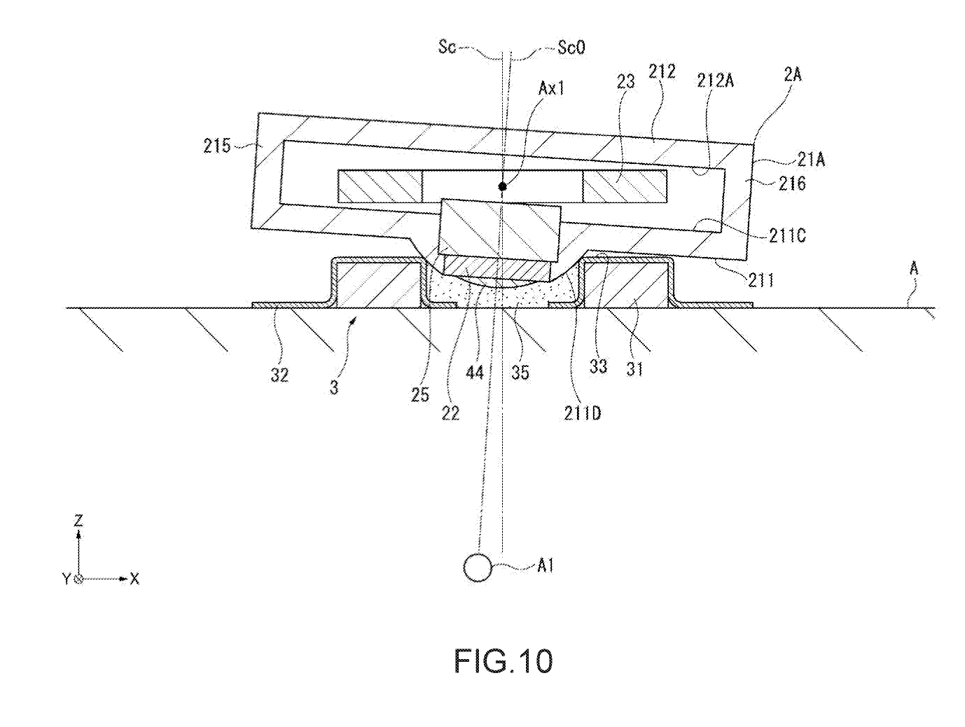

FIG. 10 is a diagram schematically showing the condition in which the angle of the scan plane of the ultrasonic probe 2A has been changed.

In the ultrasonic device according to the present embodiment, after fixing the ultrasonic probe 2A to the fixation section 3, the angle of the scan plane can be changed in the X-Z plane. Specifically, it is possible to drive the rotary mechanism 26 based on the control by the control device 5 to rotate the probe-side magnet 23 around the shaft center Ax1 relatively to the housing 21 (the ultrasonic device 22). For example, as shown in FIG. 10, when rotating the probe-side magnet 23 counterclockwise relatively to the housing 21, since the probe-side magnet 23 and the fixation section-side magnet attract each other, the housing 21 rotates clockwise relatively to the fixation section 3 while the curved contact surface 211D and a part of the probe-opposed surface 33 of the fixation section 3 keep having contact with each other. Therefore, the scan plane Sc is tilted relatively to the scan plane Sc0 corresponding to the probe-side magnet 23, which has not yet rotated.

Thus, it is possible to adjust the angle of the scan plane Sc so that the measurement object A1 is located on the scan plane Sc.

### Functions and Advantages of Second Embodiment

In the present embodiment, in addition to the functions and advantages in the first embodiment described above, the following functions and advantages can be obtained. Specifically, by rotating the probe-side magnet 23 using the rotary mechanism 26, it is possible to rotate the ultrasonic device 22 in the opposite direction to the rotational direction of the probe-side magnet 23. Therefore, the transmission direction of the ultrasonic wave of the ultrasonic device 22 and the reception direction of the ultrasonic wave, in which the ultrasonic wave can be received with high sensitivity, can be rotated around the rotational axis. Therefore, even in the case in which the transmission direction (or the reception direction of the ultrasonic wave) of the ultrasonic wave is shifted from the transmission target (or the transmission (reflection) position of the ultrasonic wave to be received) of the ultrasonic wave in the object, it is possible to easily adjust the angle of the ultrasonic device 22 with respect to the object using the rotary mechanism 26.

Further, in the present embodiment, the ultrasonic device 22 has the one-dimensional array structure in which the ultrasonic transducer groups 45A are arranged in the Y direction (the scanning direction), and is capable of controlling the transmission direction of the ultrasonic wave along the scan plane Sc parallel to the Y-Z plane and perpendicular to the operating surface 41B. Further, it is possible for the rotary mechanism 26 to rotate the probe-side magnet 23 around the rotational axis parallel to the Y direction to thereby rotate (tilt) the scan plane Sc. Therefore, even in the case in which the transmission direction of the ultrasonic wave and the transmission target of the ultrasonic wave in the object are shifted from each other along the second direction, the shift can easily be corrected by the rotary mechanism.

Further, since the rotary mechanism 26 rotates the probe-side magnet 23, it is possible to suppress the stress applied from the rotary mechanism to the ultrasonic device compared to the case of rotating the ultrasonic device 22 using the rotary mechanism 26, and thus, the degradation of the transmission characteristics and the reception characteristics of the ultrasonic wave can be suppressed.

Further, in the present embodiment, the contact surface 211D of the ultrasonic probe 2 is curved convexly toward the −Z side. Therefore, in the case in which the ultrasonic device 22 is fixed to the housing 21 of the ultrasonic probe 2 provided with the contact surface 211D, and it is arranged that the probe-side magnet 23 is rotatable with respect to the housing 21, when rotating the probe-side magnet 23 relatively to the ultrasonic device 22, the ultrasonic device 22 and the housing 21 rotate around the rotational axis. On this occasion, in the present embodiment, since the contact surface 211D is curved convexly, it is possible to smoothly rotate the housing 21 in the state in which a part of the housing 21 keeps having contact with the fixation section 3 during the rotation of the housing 21, and thus, the smooth angle adjustment can be achieved.

### Modified Example of Second Embodiment

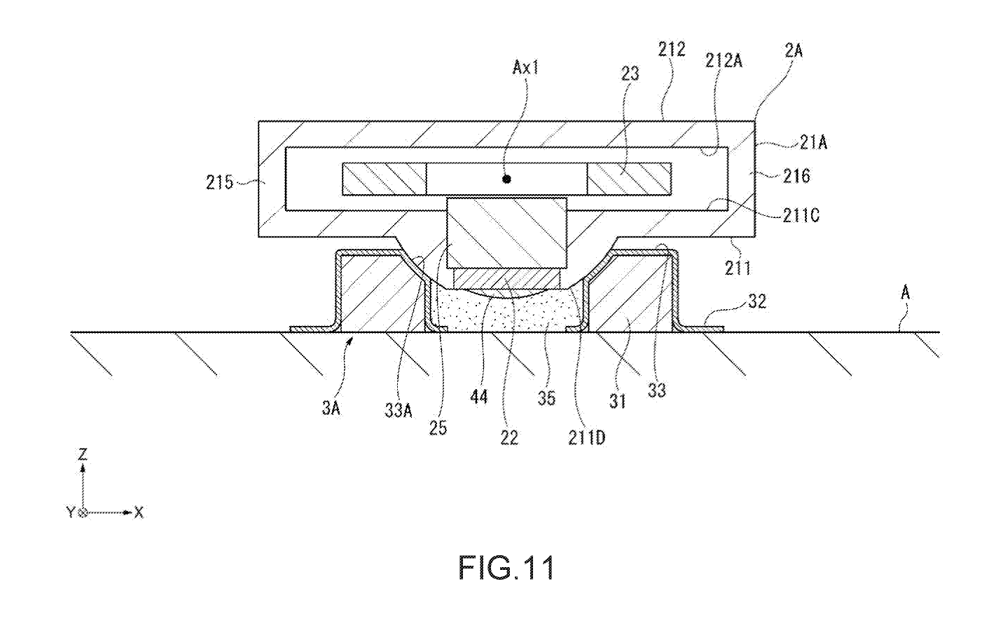

It should be noted that in the second embodiment described above, the contact surface 211D of the ultrasonic probe 2 has contact with straight line parts extending along the inner ends (opening ends) of the frame in the probe-opposed surface 33 of the fixation section 3 having the rectangular frame shape as shown in FIG. 8 and FIG. 10. In contrast, it is also possible to adopt a configuration in which the probe-opposed surface 33 of the fixation section 3A has surface contact with the contact surface 211D as shown in FIG. 11.

FIG. 11 is a schematic cross-sectional view of the ultrasonic probe 2A according to the modified example of the second embodiment and the fixation section 3A in the case of fixing the ultrasonic probe 2A to the body surface of the living body.

In the present embodiment, similarly to the first and second embodiments described above, the fixation section 3A is provided with the fixation section-side magnet 31 formed to have a rectangular frame shape in the planar view viewed from the Z direction, and a fixation tape 32.

Here, the fixation section-side magnet 31 is provided with curved surfaces forming circular arcs on the inner side (opening part side) of the surface opposed to the ultrasonic probe 2A in the Z-X plane as shown in FIG. 11. Thus, probe-contact surfaces 33A each having the same curvature as that of the curved surface described above are also formed in the probe-opposed surface 33 of the fixation section 3A. The probe-contact surfaces 33A have the same curvature radius as that of the contact surface 211D of the ultrasonic probe 2A. Therefore, when fixing the ultrasonic probe 2A to the fixation section 3A, the contact surface 211D of the ultrasonic probe 2A has contact with the probe-contact surface 33A of the fixation section 3A.

Therefore, in the present embodiment, when rotating the probe-side magnet 23 to rotate the housing 21 around the shaft center Ax1 after fixing the ultrasonic probe 2A to the fixation section 3A, the contact surface 211D has sliding contact with the probe-contact surface 33A of the fixation section 3A.

In such a configuration, the contact area in the case of fixing the fixation section 3A and the ultrasonic probe 2A to each other can be made larger compared to the second embodiment, and thus, stronger fixation force can be obtained. Further, the leakage of the acoustic matching material 35, with which the space between the opening part (the space in the frame) of the fixation section 3A, the living body A, and the ultrasonic probe 2A is filled, can be suppressed.

### Third Embodiment

Then, a third embodiment will be described.

In the first embodiment described above, the ultrasonic probe 2 is configured so that the scan plane to the body surface can be displaced along the X direction by displacing the probe-side magnet 23 along the X direction relatively to the housing 21. Further, in the second embodiment described above, there is adopted a configuration in which the angle of the scan plane to the body surface can be changed by rotating the probe-side magnet 23 with respect to the housing 21. In contrast, in the present embodiment, similarly to the first embodiment described above, the probe-side magnet 23 is disposed so as to be movable relatively to the housing, and at the same time, the ultrasonic device 22 is configured so as to be rotatable with respect to the housing, and thus, there is adopted a configuration in which the relative position along the X direction and the angle of the ultrasonic device 22 and the probe-side magnet 23 can be changed. In this point, the present embodiment is different from the first and second embodiments described above.

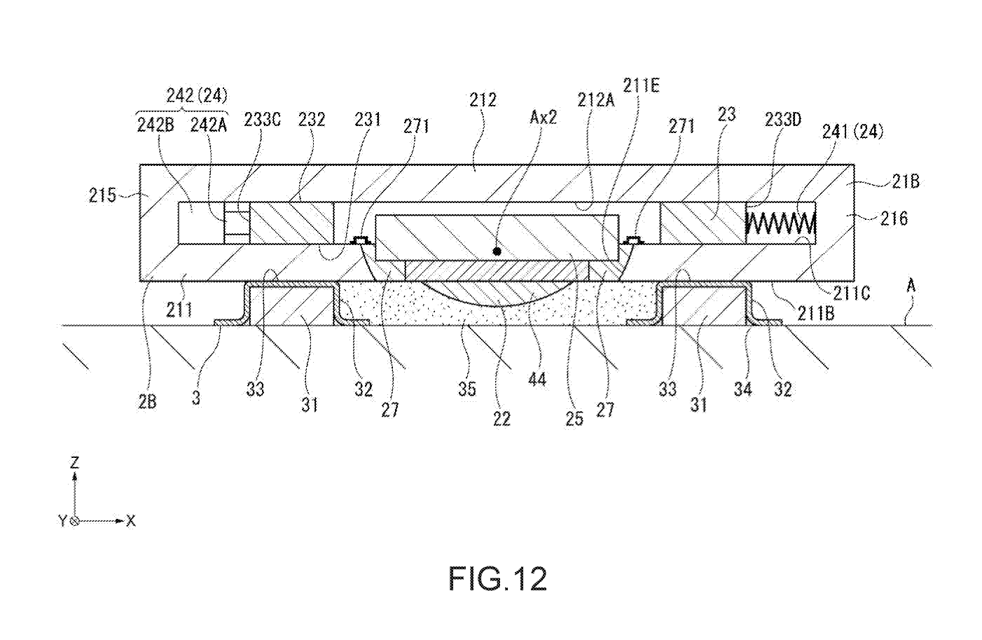

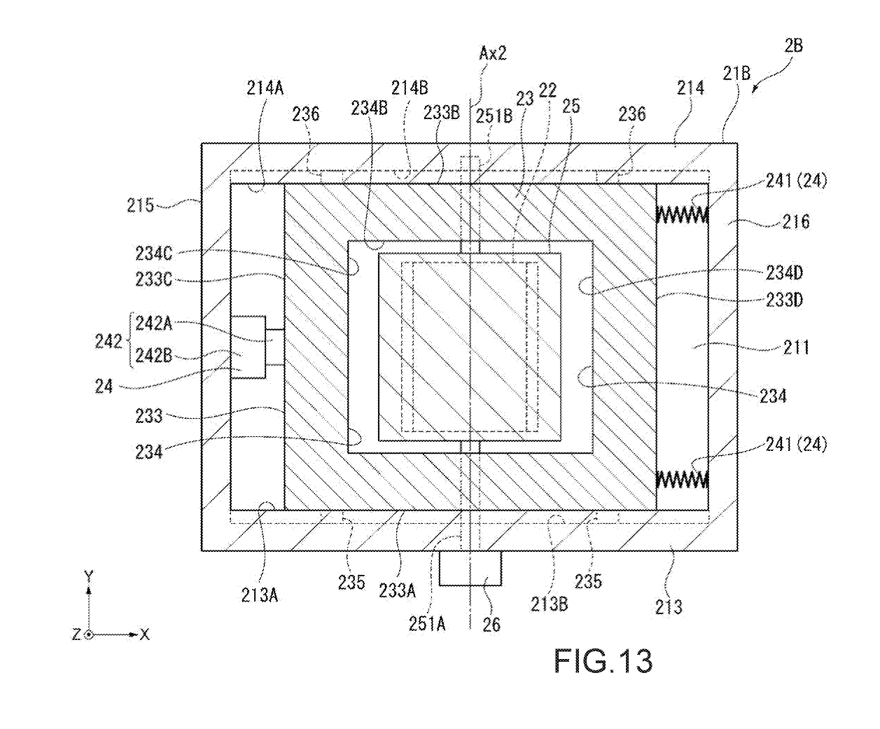

FIG. 12 is a schematic cross-sectional view of the ultrasonic probe 2B according to the third embodiment and the fixation section 3 in the case of fixing the ultrasonic probe 2B to the body surface. FIG. 13 is a schematic cross-sectional view showing a cross-section of the ultrasonic probe 2B along the X-Y plane.

Similarly to the first embodiment, the ultrasonic probe 2B is provided with the displacement mechanism 24, and is configured so that the probe-side magnet 23 can be displaced along the X direction.

Here, in the ultrasonic probe 2B according to the present embodiment, the ultrasonic device 22 is rotatably supported with respect to the housing 21B via the wiring board 25. Specifically, the wiring board 25 is supported by the housing 21B with rotary shafts 251A, 251B shown in FIG. 12 so as to be rotatable around the center axis (a shaft center Ax2) parallel to the Y direction as shown in FIG. 12. In other words, in the present embodiment, the adjustment section according to the invention is configured including the displacement mechanism 24 and the rotary mechanism 26.

The rotary mechanism 26 is connected to the rotary shaft 251A, and rotates the rotary shaft 251A to thereby rotate the wiring board 25 relatively to the housing 21B. Since the ultrasonic device 22 is fixed to the wiring board 25, the ultrasonic device 22 is also rotated in accordance with the rotation of the wiring board 25. It should be noted that although in the present embodiment, there is shown an example in which the wiring board 25 is rotatably supported by the rotary shafts 251A, 251B, but the invention is not limited to this example. It is also possible for the ultrasonic device 22 to rotatably be held directly by the rotary shafts 251A, 251B. In this case, by connecting the rotary shafts 251A, 251B to the sealing plate 42 for reinforcing the element substrate 41, stress transmission to the element substrate 41 can be suppressed. Further, although there is shown an example in which the wiring board 25 is held by the rotary shafts 251A, 251B, the invention is not limited to this example, but it is also possible to adopt a configuration in which, for example, the wiring board 25 is rotatably supported by a single rotary shaft.

In the bottom surface part 211 of the housing 21B, there is formed a sensor window 211E. In the present embodiment, the sensor window 211E is a through hole penetrating the bottom surface part 211 along the Z direction, and wall parts on the ±X side are curved along the rotational range of the ultrasonic device 22 and the wiring board 25. Specifically, the inner surfaces of the sensor window 211E are formed to be curved surfaces each having a circular arc shape centered on the shaft center Ax2 of the rotary shafts 251A, 251B in the Z-X plane.

Further, in the present embodiment, the ultrasonic device 22 and the wiring board 25 are held by a holding frame 27. As shown in FIG. 12, the holding frame 27 has outer peripheral surfaces along the inner surfaces (the curved surfaces each having the circular arc shape centered on the shaft center Ax2 in the Z-X plane) of the sensor window 211E, and becomes rotatable around the shaft center Ax2 together with the ultrasonic device 22 and the wiring board 25. Further, on the inner bottom surface 211C of the bottom surface section 211, there are disposed flexible waterproof sheets 271 covering the boundaries between the inner bottom surface 211C and the holding frame 27. Thus, there is reduced the possibility that the acoustic matching material 35 such as gel enter the inside of the housing 21B.

In such a configuration, it is possible to rotate the ultrasonic device 22 and the wiring board 25 without interfering with the housing 21B. It should be noted that the housing 21B is configured similarly to the first embodiment except the point that the wall parts of the sensor window 211A are curved.

In the ultrasonic apparatus 1 according to the third embodiment having such a configuration as described above, similarly to the first embodiment, by displacing the probe-side magnet 23 along the X direction, it is possible to move the scan plane by the ultrasonic device 22 along the X direction relatively to the body surface. Further, in the ultrasonic apparatus 1, by rotating the wiring board 25 to which the ultrasonic device 22 is fixed, the angle of the scan plane can be changed.

### Functions and Advantages of Third Embodiment

In the ultrasonic probe 2B according to the present embodiment, the ultrasonic device 22 becomes rotatable around the rotary shafts 251A, 251B, which are parallel to the Y direction, and at the same time, the probe-side magnet 23 becomes movable along the X direction relatively to the housing 21B. Therefore, similarly to the first and second embodiments described above, even after fixing the ultrasonic probe 2B to the fixation section 3, it is possible to adjust the transmission/reception direction of the ultrasonic wave so that the measurement object A1 is located on the scan plane Sc.

On this occasion, in the present embodiment, the positioning of the scan plane Sc can be performed using both of the translation of the scan plane Sc toward the X direction and rotation of the scan plane Sc centered on the shaft center Ax2.

In this case, compared to the case in which the positioning of the scan plane Sc is performed using, for example, only the displacement of the probe-side magnet 23 toward the X direction, or only the rotation of the ultrasonic device 22, the adjustment amount of each of the displacement and the rotation can be made smaller, and further, finer positioning control of the scan plane Sc can be achieved. Further, even in the case in which the measurement object is located on the −Z side of a part as a non-measurement object, by setting the angle of the scan surface Sc so as to cross the Z axis, and performing the positioning, it is possible to perform transmission and reception of the ultrasonic wave around the non-measurement object.

### Modified Examples

It should be noted that the invention is not limited to each of the embodiments described above, but includes modifications and improvements within a range where the advantages of the invention can be achieved, and configurations, which can be obtained by, for example, arbitrarily combining the embodiments.

For example, in the first and third embodiments described above, there is shown the example in which the two biasing sections 241 are disposed as the displacement mechanism 24, but the invention is not limited to this example. For example, three or more biasing sections can also be disposed, or there can also adopted a configuration of disposing a single biasing section at the central part in the Y direction.

Further, as the biasing section 241, it is also possible to use an extension coil spring, which is disposed on the −X side of the probe-side magnet 23 and pulls the probe-side magnet 23 toward the −X side, and so on. Further, it is also possible to adopt a configuration in which a pressing member such as a compression spring for pressing the probe-side magnet 23 toward the −X side is disposed on the +X side of the probe-side magnet 23, and the tensile member for pulling the probe-side magnet 23 toward the −X side is disposed on the −X side of the probe-side magnet 23.

Further, although there is illustrated the configuration of moving the spindle forward and backward using the advancing/retreating mechanism of the micrometer head as the pressing part 242A and the drive section 242B of the pressing section 242, it is also possible to adopt a configuration of moving a pin forward and backward using an actuator such as a solenoid.

Further, although there is shown the example of pressing the probe-side magnet 23 with the pressing section 242, it is also possible to adopt a configuration of fixing the probe-side magnet 23 to the tip of a shaft which can be driven forward and backward by a configuration such as a solenoid, and so on. In this case, the configuration of the biasing section 241 can be made unnecessary.

Further, as the displacement mechanism 24, there can be used a variety of types of actuators as described above. It is possible to illustrate a configuration of using, for example, a piezoelectric actuator, which causes an elliptic motion in the tip part to thereby move a surface having contact with the tip part along the X direction, on at least either one of the first outer side surface 233A, the second outer side surface 233B, the first inner side surface 234A, and the second inner side surface 234B of the probe-side magnet 23. Further, instead of the piezoelectric actuator, it is also possible to use a surface-wave type ultrasonic motor, which propagates the surface wave along the X direction to move the probe-side magnet 23, or the like on at least either one of the first outer side surface 233A, the second outer side surface 233B, the first inner side surface 234A, and the second inner side surface 234B of the probe-side magnet 23. Further, it is also possible to adopt a configuration of rotating a screw shaft extending along the X direction by an electric motor, and engaging a part of the probe-side magnet 23 with the screw shaft.

Further, in the first and third embodiments described above, there is illustrated the configuration guiding the displacement of the probe-side magnet 23 by making the probe-side magnet 23 have sliding contact with the wall surfaces (the first side surface part 213 and the second side surface part 214) of the housing 21, 21B, and at the same time, inserting the protrusions (the first protruding parts 235 and the second protruding parts 236) of the probe-side magnet 23 into the groove parts (the first groove part 213B and the second groove part 214B) provided to the wall surfaces, but the invention is not limited to this configuration. For example, it is also possible to adopt a configuration of making a shaft disposed along the X direction hold the probe-side magnet 23. Further, it is also possible to adopt a configuration of providing the probe-side magnet 23 with a through hole extending along the X direction, and inserting a shaft fixed to the housing into the through hole, or the like. In this case, it is also possible to make the shaft hold the coil spring constituting the biasing section 241.

Further, as the rotary mechanism 26 in the second and third embodiments, there is illustrated a stepping motor, but the invention is not limited to this example. It is also possible to adopt a configuration of, for example, making a piezoelectric actuator with the tip part performing an elliptic motion have contact with a rotor disposed at the tip of each of the rotary shafts 237A, 237B, 251A, 251B to thereby rotate the rotary shafts 237A, 237B, 251A, 251B.

Further, in the second embodiment, there is illustrated the configuration of rotating the probe-side magnet 23 around the rotary shafts 237A, 237B as the rotary mechanism 26, but the invention is not limited to this configuration.

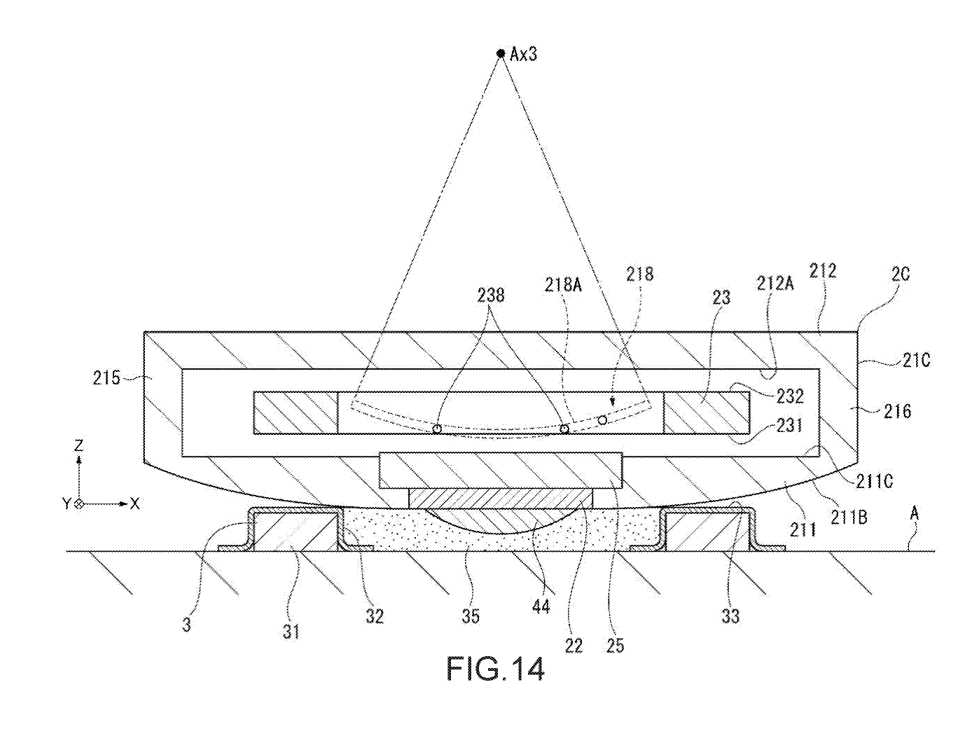

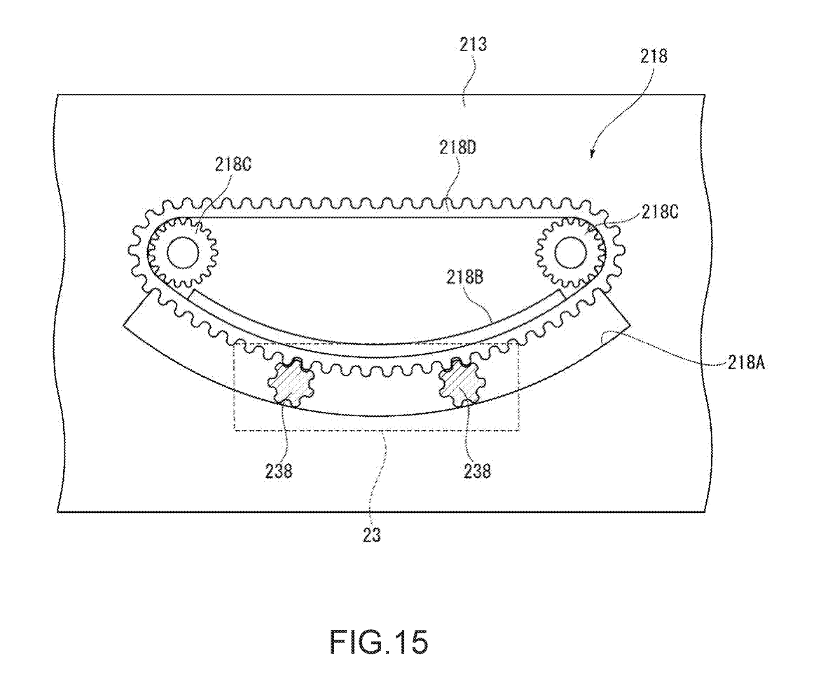

It is also possible to adopt a configuration of, for example, swinging the probe-side magnet 23 along a circular arc groove as shown in FIG. 14 and FIG. 15.

FIG. 14 is a schematic cross-sectional view of an ultrasonic probe 2C according to the present modified example and a fixation section 3 in the case of fixing the ultrasonic probe 2C to the body surface.

FIG. 15 is a diagram for explaining an outline of a swing mechanism (an adjustment section) of the probe-side magnet 23 in the present modified example.

As shown in FIG. 15, in this ultrasonic probe 2C, there is provided the swing mechanism 218 constituting the adjustment section according to the invention. The swing mechanism 218 is constituted by guide grooves 218A, belt guides 218B, pulleys 218C, belts 218D, and pinion gears 238. It should be noted that in FIG. 14, illustration of the guide groove 218A, the belt guide 218B, the pulleys 218C, the belt 218D, and the pinion gears 238 on the second side surface part 214 side is omitted.

The guide grooves 218A are grooves each having a circular arc shape and are provided respectively to the first side surface part 213 and the second side surface part 214 of the housing 21C. As shown in FIG. 14, the circular arc center (an imaginary shaft center Ax3) of the guide groove 218A is disposed outside the ultrasonic probe 2C.

The belt guides 218B are disposed along the circular arc of the guide grooves 218A on the +Z side (the top surface part 212 side) of the guide grooves 218A of the first side surface part 213 and the second side surface part 214, respectively. Further, the pulleys 218C are disposed on both end sides in the X direction of the belt guides 218B so as to be able to rotate around the rotary shaft perpendicular to the first side surface part 213 (and the second side surface part 214).

Further, the belts 218D are each wound around the pair of pulleys 218C, and a part of the belt 218D located on the −Z side has sliding contact with a surface on the −Z side of the belt guide 218B. Further, the outer peripheral surface of the belt 218D is provided with rack grooves as shown in FIG. 15.

One of the pair of pulleys 218C becomes to be able to be rotationally driven by a drive section not shown, and thus, it becomes possible to drive the −Z side of the belt 218D along the guide groove 218A.

Further, the pinion gears 238 protrude from the probe-side magnet 23 toward the first side surface part 213 (and the second side surface part 214), and are inserted into the guide grooves 218A. Further, the pinion gears 238 engage with the rack grooves provided to the outer peripheral surface of the belt 218D, and when the pulleys 218C are rotationally driven to move the belts 218D along the guide grooves 218A, the pinion gears 238 rotate to move along the guide grooves 218A. Thus, the probe-side magnet 23 becomes possible to swing along the guide grooves 218A together with the pinion gears 238.

In such an ultrasonic probe 2C according to the modified example as described above, by swinging the probe-side magnet 23 in the state in which the relative position to the fixation section-side magnet 31 is fixed, the housing 21C is displaced in the direction along the guide grooves 218A. Thus, the angle and the position in the X direction of the scan plane Sc of the ultrasonic wave transmitted and received by the ultrasonic device 22 are changed. Thus, it becomes possible to adjust the fixation position of the ultrasonic probe 2C so that the measurement object A1 is located on the scan plane Sc.

It should be noted that the swing mechanism is not limited to the example shown in FIG. 15. It is also possible to adopt a configuration in which, for example, rack grooves are formed on the inner peripheral surface of the guide groove 218A, the pinion gears 238 are rotationally driven by a stepping motor provided to the probe-side magnet 23 to thereby swing the probe-side magnet 23.

Further, although there is illustrated the configuration of swinging the probe-side magnet 23 relatively to the housing 21C, it is also possible to adopt a configuration of swinging the ultrasonic device 22 relatively to the housing 21C. The configuration can be realized with substantially the same configuration as the swing mechanism 218 of the probe-side magnet 23, and it is sufficient to, for example, provide the ultrasonic device 22 with the pinion gears to be inserted into the guide grooves 218A.

In the first embodiment, there is shown the example in which the ultrasonic device 22 is fixed to the housing 21, and by displacing the probe-side magnet 23 relatively to the housing 21, the housing is displaced in the X direction relatively to the fixation section 3, but the invention is not limited to this example.

It is also possible to adopt a configuration in which, for example, the probe-side magnet 21 is fixed to the housing 21, and the ultrasonic device 22 becomes to be able to be displaced relatively to the housing 21, and displaced along the X direction by the displacement mechanism.

Further, the same applies to the second embodiment, it is also possible to adopt a configuration in which, for example, the probe-side magnet 23 is fixed to the housing 21, and the ultrasonic device 22 becomes rotatable around the rotary shaft extending along the Y direction with respect to the housing 21 as in the third embodiment.

Further, also in the third embodiment, it is also possible to adopt a configuration in which the probe-side magnet 23 is rotatable around the rotary shaft extending along the Y direction with respect to the housing 21 to thereby make the ultrasonic device 22 movable in the X direction using the displacement mechanism 24.

In each of the embodiments described above, the bulk type probe-side magnet 23 is illustrated as the first fixation section according to the invention, but the invention is not limited to this example. It is also possible to use, for example, a coil magnet.

Further, as the first fixation section and the second fixation section, there is described the example of fixing the ultrasonic probe 2, 2A, 2B to the fixation section 3, 3A with the magnetic force of the magnet, but the invention is not limited to this configuration.

It is also possible to adopt a configuration in which, for example, a click part (a male engagement part) is provided to either one of the ultrasonic probe 2 and the fixation section 3, an engagement part (a female engagement part) is provided to the other thereof, and the click part is made to engage with the engagement part. In this case, by adopting a configuration of, for example, displacing the click part relatively to the housing to which the ultrasonic device is fixed, it is possible to displace the scan plane along the X direction similarly to each of the embodiments described above.

Besides the above, it is also possible to adopt a configuration in which an adhesive surface is provided to the probe-opposed surface 33 of the fixation section, and the ultrasonic probe 2 is adhesively fixed, and so on. In this case, by, for example, displacing the ultrasonic device 22 relatively to the housing 21, it is possible to displace the scan plane along the X direction similarly to each of the embodiments described above.

In each of the embodiments described above, there is illustrate the configuration in which the fixation section 3 has the frame-like shape, but the invention is not limited to this configuration.

It is also possible to adopt a configuration of, for example, fixing two fixation section-side magnets 31 parallel to each other to the body surface of the living body A with the fixation tape 32.

Further, although there is shown the example in which the fixation section 3 is constituted by the fixation section-side magnet 31 and the fixation tape 32, it is also possible to adopt a configuration in which, for example, the fixation surface 34 of the fixation section-side magnet 31 is an adhesive surface, and can directly fixed adhesively to the living body A.

In the embodiments described above, there is shown the example in which the ultrasonic device 22 has the one-dimensional structure having the groups of the ultrasonic transducers 45 arranged along the Y direction, but the invention is not limited to this structure. It is also possible to adopt a configuration in which the plurality of ultrasonic transducers 45 is arranged in the X direction and the Y direction to constitute a two-dimensional structure. In this case the acoustic lens 44 is not required to be provided.

In the second and third embodiments, there is shown the example in which the rotary shafts 237A, 237B, 251A, and 251B are disposed in parallel to the Y direction, but the invention is not limited to this example. For example, if the rotary shafts 237A, 237B cross the X direction, which is an arrangement direction of the ultrasonic transducers in each of the ultrasonic transducer groups 45A, by rotating either one of the ultrasonic device 22 and the probe-side magnet 23 around the rotary shafts 237A, 237B, 251A, and 251B, it is possible to swing the scan plane Sc in the X direction.

In the first embodiment, there is shown the example in which the contact surface 211B of the ultrasonic probe 2 and the probe-opposed surface 33 of the fixation section 3 are each a roughly flat surface, but the invention is not limited this example. It is also possible to adopt a configuration in which, for example, a groove extending along the X direction is provided to one of the contact surface 211B and the probe-opposed surface 33, and a protruding part engaging with the groove is provided to the other. In such a configuration, the displacement of the ultrasonic probe 2 in the direction crossing the X direction can be limited.

In the first embodiment, there is shown the example of displacing the probe-side magnet 23 along the X direction, but it is also possible to adopt a configuration of displacing the probe-side magnet 23 in a direction tilted (except the right angle) with respect to, for example, the X direction. Further, there is shown the example of displacing the probe-side magnet 23 along the X-Y plane, but it is also possible to adopt a configuration of displacing the probe-side magnet 23 in a direction tilted (except the right angle) with respect to, for example, the X-Y plane. In other words, in the invention, the relative movement direction between the first fixation section and the ultrasonic probe is not limited to the X direction, but it is also possible to perform the relative movement with a tilt toward a direction except the right angle direction with respect to the X direction. Also in this case, the scan plane Sc can be displaced in the X direction with the displacement amount corresponding to the X direction component.

In each of the embodiments, there is illustrated the configuration in which the lower-part electrode 414, the piezoelectric film 415, and the upper-part electrode 416 are stacked on the vibrating film 412 as the ultrasonic transducer 45, but the invention is not limited to this configuration. It is also possible to adopt a configuration in which, for example, a pair of electrodes are disposed on one surface perpendicular to the thickness direction of the piezoelectric element 413 so as to be opposed to each other as the ultrasonic transducer. Further, it is also possible to dispose the electrodes on the side surfaces along the thickness direction of the piezoelectric film so as to sandwich the piezoelectric film.

Further, as the ultrasonic transducer, it is also possible to vibrate the vibrating film or detect the vibration of the vibrating film using a drive method other than the piezoelectric method using the piezoelectric film. It is possible to illustrate a configuration in which, for example, the first electrode is disposed on the substrate, the vibrating film is disposed on the first electrode with a predetermined air gap, and the second electrode opposed to the first electrode is disposed on the vibrating film. In this case, by applying the pulse drive voltage between the first electrode and the second electrode, it is possible to drive the vibrating film with the electrostatic force to output the ultrasonic wave, and when the vibrating film vibrates due to the received ultrasonic wave, the capacitance between the first electrode and the second electrode varies, and therefore, the reception of the ultrasonic wave can be detected based on the variation of the capacitance.

In the first embodiment, there is illustrated the ultrasonic measurement apparatus for transmitting the ultrasonic wave to the living body A, and receiving the ultrasonic wave reflected by the measurement object A1 in the living body A to thereby measure the internal structure of the living body A as the ultrasonic apparatus of the invention, but the invention is not limited to this example.

For example, the object is not limited to the living body A, but it is also possible to adopt an apparatus for performing the ultrasonic measurement on other objects such as a concrete structure.

Further, the invention is not limited to the ultrasonic measurement apparatus, but can also be, for example, a transmission-only ultrasonic apparatus for transmitting the ultrasonic wave to the object. As such an ultrasonic apparatus, there can be illustrated, for example, an ultrasonic therapy apparatus for transmitting the ultrasonic wave to an affected part of the living body.

Besides the above, specific structures to be adopted when implementing the invention can be configured by arbitrarily combining the embodiments and the modified examples described above with each other, or can arbitrarily be replaced with other structures and so on within the range in which the advantages of the invention can be achieved.

The entire disclosure of Japanese Patent Application No. 2015-235448, filed Dec. 2, 2015 is expressly incorporated by reference herein.

What is claimed is:

1. An ultrasonic probe unit comprising:
an ultrasonic probe; and
a fixation section adapted to fix the ultrasonic probe to an object,
wherein the ultrasonic probe includes
an ultrasonic device adapted to perform at least one of transmission and reception of an ultrasonic wave,
a first fixation section, and
an adjustment section adapted to adjust a relative positional relationship between the ultrasonic device and the first fixation section, and
the fixation section includes
a fixation surface to be fixed to the object, and
a second fixation section a relative position of which to the first fixation section is fixed to thereby fix the ultrasonic probe to the object.

2. The ultrasonic probe unit according to claim 1, wherein
the adjustment section has a displacement mechanism adapted to displace the first fixation section relatively to the ultrasonic device.

3. The ultrasonic probe unit according to claim 2, wherein
the ultrasonic device has a device surface, which is at least one of a surface adapted to transmit the ultrasonic wave and a surface adapted to receive the ultrasonic wave, and
the displacement mechanism displaces the first fixation section relatively to the ultrasonic device in a plane parallel to the device surface.

4. The ultrasonic probe unit according to claim 3, wherein
the ultrasonic device has a one-dimensional array structure in which a plurality of ultrasonic transducers is arranged along a first direction parallel to the device surface, and
the displacement mechanism displaces the first fixation section relatively to the ultrasonic device along a second direction crossing the first direction.

5. The ultrasonic probe unit according to claim 2, wherein
the ultrasonic device has a device surface, which is at least one of a surface adapted to transmit the ultrasonic wave and a surface adapted to receive the ultrasonic wave,
the ultrasonic probe has a contact surface having contact with the fixation section and parallel to the device surface, and
the fixation section has a plane along the contact surface.

6. The ultrasonic probe unit according to claim 2, wherein
the displacement mechanism displaces the first fixation section.

7. The ultrasonic probe unit according to claim 1, wherein
the ultrasonic device has a device surface, which is at least one of a surface adapted to transmit the ultrasonic wave and a surface adapted to receive the ultrasonic wave, and
the adjustment section includes a rotary mechanism adapted to rotate at least one of the ultrasonic device and the first fixation section around a rotational axis crossing a normal line of the device surface.

8. The ultrasonic probe unit according to claim 7, wherein
the ultrasonic device has a one-dimensional array structure in which a plurality of ultrasonic transducers is arranged along a first direction parallel to the device surface, and
the rotational axis is parallel to the first direction.

9. The ultrasonic probe unit according to claim 7, wherein
the rotary mechanism rotates the ultrasonic device.

10. The ultrasonic probe unit according to claim 7, wherein
the rotary mechanism rotates the first fixation section.

11. The ultrasonic probe unit according to claim 10, wherein
the ultrasonic probe has a contact surface having contact with the fixation section, and
the contact surface is curved convexly toward a direction from the device surface toward the fixation section when being cut by a plane perpendicular to the rotational axis.

12. The ultrasonic probe unit according to claim 11, wherein
the fixation section has a curved surface curved along the contact surface.

13. The ultrasonic probe unit according to claim 1, wherein
the ultrasonic probe has an acoustic lens protruding from a contact surface having contact with the fixation section toward the object.

14. The ultrasonic probe unit according to claim 13, wherein
the fixation section has a frame body adapted to house the acoustic lens.

15. The ultrasonic probe unit according to claim 1, wherein
the first fixation section and the second fixation section are each a magnet.

16. An ultrasonic probe to be fixed to a fixation section fixed to an object, comprising:
an ultrasonic device adapted to perform at least one of transmission and reception of an ultrasonic wave;
a first fixation section a relative position of which to a second fixation section provided to the fixation section is fixed to thereby fix the ultrasonic probe to the fixation section; and
an adjustment section adapted to adjust a relative positional relationship between the ultrasonic device and the first fixation section.

17. An ultrasonic apparatus comprising:
an ultrasonic probe;
a fixation section adapted to fix the ultrasonic probe to an object; and
a control section adapted to control the ultrasonic probe,
wherein the ultrasonic probe includes
an ultrasonic device adapted to perform at least one of transmission and reception of an ultrasonic wave,
a first fixation section, and
an adjustment section adapted to adjust a relative positional relationship between the ultrasonic device and the first fixation section, and
the fixation section includes
a fixation surface to be fixed to the object, and
a second fixation section a relative position of which to the first fixation section is fixed to thereby fix the ultrasonic probe to the object.
